(19) United States
(12) Patent Application Publication
(10) Pub. No.: US 2024/0369925 A1
(43) Pub. Date: Nov. 7, 2024

(54) RESIST AUXILIARY FILM COMPOSITION, AND PATTERN FORMING METHOD USING SAID COMPOSITION

(71) Applicant: MITSUBISHI GAS CHEMICAL COMPANY, INC., Tokyo (JP)

(72) Inventors: Takumi OKADA, Niigata (JP); Ryosuke HOSHINO, Niigata (JP); Hideyuki SATO, Niigata (JP); Masayuki KATAGIRI, Niigata (JP); Shu SUZUKI, Kanagawa (JP); Masatoshi ECHIGO, Tokyo (JP)

(73) Assignee: MITSUBISHI GAS CHEMICAL COMPANY, INC., Tokyo (JP)

(21) Appl. No.: 18/291,760

(22) PCT Filed: Jul. 25, 2022
(86) PCT No.: PCT/JP2022/028577

§ 371 Date: Jan. 24, 2024

(30) Foreign Application Priority Data
Jul. 30, 2021 (JP) 2021-125373
Sep. 10, 2021 (JP) 2021-147669

(51) Int. Cl. G03F 7/004; G03F 7/00; G03F 7/075; G03F 7/11

(52) U.S. Cl. CPC G03F 7/0048; G03F 7/0035; G03F 7/0045; G03F 7/075; G03F 7/11

(57) ABSTRACT

A resist auxiliary film composition includes: (A) a resin; and (B) a solvent containing: (B1) a compound represented by the following general formula (b-1), wherein the content of the active component is 45% by mass or less based on the total amount of the resist auxiliary film composition:

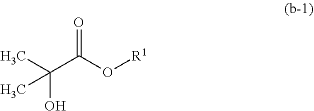

wherein R¹ is an alkyl group having 1 to 10 carbon atoms.

### TECHNICAL FIELD

The present invention relates to a resist auxiliary film composition, and a method for forming a pattern using the composition.

### BACKGROUND ART

In recent years, along with increases in the integration and speed of semiconductor devices, miniaturization of pattern rules has been required. In such a situation, various technical developments have been made in regard to how processing is performed to obtain finer and more precise pattern taking the light source to be used into consideration, in lithography involving exposure to light, which is currently used as a general-purpose technique.

Regarding the light source for lithography used for resist pattern formation, exposure to light of g-line (436 nm) or i-line (365 nm) of a mercury lamp as the light source is widely used for a portion where the integration degree is low. On the other hand, for a portion where the integration degree is so high that miniaturization is required, lithography with KrF excimer laser (248 nm) or ArF excimer laser (193 nm), which has a shorter wavelength, has also been put into practical use. In the state-of-the-art generation, which requires further miniaturization, lithography with extreme ultraviolet (EUV, 13.5 nm) is coming into practical use. In addition, various resist auxiliary films for improving the performance of a photoresist are used to improve miniaturization.

Along with the application of KrF excimer laser and ArF excimer laser, diffused reflection of active rays from a substrate and the influence of standing waves have been large problems, so that a method has widely been employed in which an antireflection film (Bottom Anti-Reflective Coating, BARC), as the resist underlayer film functioning to prevent reflection, is provided between a photoresist and a substrate to be processed.

Known as the antireflection film are inorganic antireflection films of, for example, titanium, titanium dioxide, titanium nitride, chromium oxide, carbon, and α-silicon, and organic antireflection films consisting of a light absorption substance and a polymer compound. The former requires equipment for film formation, such as a vacuum vapor deposition apparatus, a CVD apparatus, or a sputtering apparatus. In contrast, the latter advantageously requires no special equipment, and many examinations have been made thereon.

Examples thereof include an antireflection film including an acrylic resin having a hydroxyl group as a cross-linking reactive group and a light absorbing group in the same molecule (see Patent Literature 1), and an antireflection film including a novolac resin having a hydroxyl group as a cross-linking reactive group and a light absorbing group in the same molecule (see Patent Literature 2).

The physical properties desired as the organic antireflection film material include a large absorbance to light or radiation, no intermixing with a photoresist layer (being insoluble in a resist solvent), no diffusion of low molecular substances from the material of the antireflection film into the top coat resist during application or heat drying, and a high dry etching rate as compared with that of the photoresist (see Non Patent Literature 1).

In the device manufacturing process involving EUV lithography, adverse effects caused by a base substrate or EUV cause the following problems, for example: the pattern of the resist for EUV lithography becomes a tailing shape or an undercut shape so that a good resist pattern having a straight shape cannot be formed; and the sensibility to EUV is so low that sufficient throughput cannot be obtained. Accordingly, although any resist underlayer film (antireflection film) having an antireflection ability is not required in the EUV lithography process, a resist underlayer film for EUV lithography is required that enables these adverse effects to be reduced to thereby form a good resist pattern having a straight shape and improve resist sensibility.

After the resist underlayer film for EUV lithography is formed, a resist is applied thereon, and accordingly, no intermixing with a resist layer (being insoluble in a resist solvent) and excellent adhesiveness with the resist are essential characteristics for the resist underlayer film for EUV lithography, as in the antireflection film.

Further, the resist pattern width is significantly fine in a generation using EUV lithography, the resist for EUV lithography is desired to be a thinner film. Thus, it is needed to largely reduce the time required for the removing process of the organic antireflection film by etching. Therefore, required is a resist underlayer film for EUV lithography capable of being used as a thin film, or a resist underlayer film for EUV lithography having high selectivity in the etching rate with respect to the resist for EUV lithography.

A single-layer resist method is used as a typical resist pattern formation method, and it is well known that, as the thinning of the resist pattern progresses as described above in that method, the ratio of a pattern height to a pattern line width (aspect ratio) is increased, and that pattern collapse is caused due to the surface tension of a developer during development. Thus, it is known that a multi-layer resist method, in which films each having different dry etching characteristics are laminated to form a pattern, is suitable for forming a pattern having a high aspect ratio on a stepped substrate. Then, the multi-layer resist methods are being developed, including a two-layer resist method in which a photoresist layer made of a silicon-containing photosensitive polymer and an underlayer made of an organic polymer containing carbon, hydrogen, and oxygen as the main constituent elements (for example, a novolac polymer) are combined (e.g., see Patent Literature 3), and a three-layer resist method, in which a photoresist layer made of an organic photosensitive polymer used in the single-layer resist method, an intermediate layer made of a silicon polymer or a silicon CVD film, and an underlayer made of an organic polymer are combined (e.g., see Patent Literature 4).

In the three-layer resist method, first, the pattern of the photoresist layer is transferred to the silicon-containing intermediate layer with a fluorocarbon-based dry etching gas. Thereafter, by using the pattern as a mask, the pattern is transferred to the organic underlayer film that contains carbon and hydrogen as the main constituent elements by dry etching with an oxygen-containing gas, and by using the resultant as a mask, the pattern is transferred to a substrate to be processed by dry etching. However, in semiconductor device manufacturing processes after 20 nm generation, phenomena such as twisting and curving are found in the organic underlayer film pattern when the pattern is transferred to a substrate to be processed by dry etching through the organic underlayer film pattern as a hard mask.

The carbon hard mask formed on the substrate to be processed is typically an amorphous carbon film prepared by a CVD method involving use of methane gas, ethane gas, acetylene gas, or the like as the material (hereinafter, CVD-C film). It is known that the amount of hydrogen atoms in the film can be significantly small in the CVD-C film, and the CVD-C film is very effective to the twisting and curving of the pattern as described above. However, it is also known that when the base substrate to be processed has a step, it is difficult to embed such a step into a flat state due to the characteristics of the CVD process. Thus, when a substrate to be processed having a step having been embedded with a CVD-C film is patterned with a photoresist, a step occurs on the surface applied with the photoresist due to the influence of the step of the substrate to be processed, which makes the film thickness of the photoresist non-uniform, and as the result, the focus margin during lithography and the pattern shape are poor.

On the other hand, it is known that, when the underlayer film formed immediately on the substrate to be processed as the carbon hard mask is formed by spin coating, the step of the stepped substrate can be advantageously embedded into a flat state. When the substrate is flattened with the underlayer film material, variation in the film thickness of the silicon-containing intermediate layer or photoresist formed on the underlayer can be suppressed, and the focus margin of the lithography can be thus enlarged to form a normal pattern.

Hence, there are demands for an underlayer film material that is capable of forming into a film by spin coating (spin-on-carbon film material), the film having high etching resistance and high flatness on a substrate to be processed upon performing the dry etching processing of the substrate to be processed, and a method for forming an underlayer film (spin-on-carbon film).

Typically, a material having a high carbon content is used for the spin-on-carbon film. When such a material having a high carbon content is used for the resist underlayer film, the etching resistance during substrate processing improves, and as the result, more precise pattern transfer is enabled. For such a spin-on-carbon film, a phenol novolac resin is well known (see e.g., Patent Literature 5). It is also known that a spin-on-carbon film formed from an acenaphthylene polymer-containing composition for a resist spin-on-carbon film exhibits good characteristics (see e.g., Patent Literature 6).

### CITATION LIST

### Patent Literature

Patent Literature 1: U.S. Pat. No. 5,919,599
Patent Literature 2: U.S. Pat. No. 5,693,691
Patent Literature 3: Japanese Patent Laid-Open No. 2000-143937
Patent Literature 4: Japanese Patent Laid-Open No. 2001-40293
Patent Literature 5: Japanese Patent Laid-Open No. 2010-15112
Patent Literature 6: Japanese Patent Laid-Open No. 2005-250434

### Non Patent Literature

Non Patent Literature 1: Proc. SPIE, Vol. 3678, 174-185 (1999).

### SUMMARY OF INVENTION

### Technical Problem

As described above, characteristics required of the photoresist auxiliary film material used in the manufacture of various devices such as semiconductor devices and liquid crystal devices are different depending on the type of devices. Therefore, a photoresist auxiliary film material capable of forming a resist auxiliary film suitable for the manufacture of various devices is required.

### Solution to Problem

The present inventors have intensively studied to solve the above problems, and as a result, have found that the above problems can be solved by a resist auxiliary film composition which contains a resin and a solvent including a compound having a specific structure and in which the content of the active component is limited to a predetermined value or less. That is, the present invention is as follows.

[1] A resist auxiliary film composition comprising:
(A) a resin, and
(B) a solvent comprising: (B1) a compound represented by the following general formula (b-1), wherein
a content of an active component is 45% by mass or less based on the total amount of the resist auxiliary film composition:

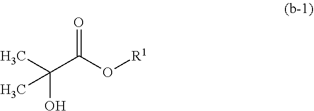

wherein R¹ is an alkyl group having 1 to 10 carbon atoms.

[2] The resist auxiliary film composition according to the above [1], further comprising: (C) at least one additive selected from the group consisting of a photosensitizer and an acid generating agent.

[3] The resist auxiliary film composition according to the above [1] or [2], wherein R¹ in the general formula (b-1) is a methyl group, an ethyl group, an n-propyl group, an i-propyl group, an n-butyl group, an i-butyl group, an s-butyl group, or a t-butyl group.

[4] The resist auxiliary film composition according to any one of the above [1] to [3], wherein R¹ in the general formula (b-1) is an ethyl group, an n-propyl group, an i-propyl group, an n-butyl group, an i-butyl group, an s-butyl group, or a t-butyl group.

[5] The resist auxiliary film composition according to any one of the above [1] to [4], wherein the solvent (B) comprises: (B2) a solvent other than the compound (B1).

[6] The resist auxiliary film composition according to the above [5], wherein the solvent (B) comprises one or more selected from the group consisting of methyl α-methoxyisobutyrate, methyl α-formyloxyisobutyrate, methyl α-acetyloxyisobutyrate, and methyl 3-hydroxyisobutyrate as the solvent (B2).

[7] The resist auxiliary film composition according to the above [5], wherein the solvent (B) comprises one or more selected from the group consisting of methyl α-methoxyisobutyrate, methyl α-formyloxyisobutyrate, methyl α-acetyloxyisobutyrate, methyl 3-hydroxyisobutyrate, and 1-methoxy-2-propanol as the solvent (B2).

[8] The resist auxiliary film composition according to any one of the above [5] to [7], wherein the solvent (B2) is contained in an amount of 100% by mass or less based on the total amount (100% by mass) of the compound (B1).

[9] The resist auxiliary film composition according to the above [8], wherein the solvent (B2) is contained in an amount of 0.0001% by mass or more based on the total amount (100% by mass) of the compound (B1).

[10] The resist auxiliary film composition according to any one of the above [5] to [9], wherein the solvent (B2) is contained in an amount less than 100% by mass based on the total amount (100% by mass) of the resist auxiliary film composition.

[11] The resist auxiliary film composition according to any one of the above [1] to [10], wherein the resin (A) comprises a novolac resin (A1).

[12] The resist auxiliary film composition according to any one of the above [1] to [10], wherein the resin (A) comprises an ethylenically unsaturated resin (A2).

[13] The resist auxiliary film composition according to any one of the above [1] to [10], wherein the resin (A) comprises a high carbon resin (A3).

[14] The resist auxiliary film composition according to any one of the above [1] to [10], wherein the resin (A) comprises a silicon-containing resin (A4).

[15] The resist auxiliary film composition according to any one of the above [1] to [14], wherein the resist auxiliary film is a resist underlayer film.

[16] The resist auxiliary film composition according to any one of the above [1] to [14], wherein the resist auxiliary film is a resist intermediate layer film.

[17] A method for forming a pattern, comprising:
step (A-1) of forming a resist underlayer film on a substrate by using the resist auxiliary film composition according to the above [15],
step (A-2) of forming at least one photoresist layer on the resist underlayer film, and
step (A-3) of irradiating a predetermined region of the photoresist layer with radiation after step (A-2), followed by developing.

[18] A method for forming a pattern, comprising:
step (B-1) of forming a resist underlayer film on a substrate by using the resist auxiliary film composition according to the above [15],
step (B-2) of forming a resist intermediate layer film on the resist underlayer film,
step (B-3) of forming at least one photoresist layer on the resist intermediate layer film,
step (B-4) of irradiating a predetermined region of the photoresist layer with radiation after step (B-3), followed by developing to form a resist pattern, and
step (B-5) of etching the resist intermediate layer film by using the resist pattern as a mask after step (B-4), etching the resist underlayer film by using the obtained resist intermediate layer film pattern as an etching mask, and etching the substrate by using the obtained resist underlayer film pattern as an etching mask to form a pattern on the substrate.

[19] A method for forming a pattern, comprising:
step (B-1) of forming a resist underlayer film on a substrate,
step (B-2) of forming a resist intermediate layer film on the resist underlayer film by using the resist auxiliary film composition according to the above [16],
step (B-3) of forming at least one photoresist layer on the resist intermediate layer film,
step (B-4) of irradiating a predetermined region of the photoresist layer with radiation after step (B-3), followed by developing to form a resist pattern, and
step (B-5) of etching the resist intermediate layer film by using the resist pattern as a mask after step (B-4), etching the resist underlayer film by using the obtained resist intermediate layer film pattern as an etching mask, and etching the substrate by using the obtained resist underlayer film pattern as an etching mask to form a pattern on the substrate.

### Advantageous Effects of Invention

The resist auxiliary film composition of a suitable aspect of the present invention can form a resist auxiliary film suitable for the manufacture of various devices though the content of the active component including the resin is limited to a predetermined value or less.

### DESCRIPTION OF EMBODIMENTS

### [Resist Auxiliary Film Composition]

The resist auxiliary film composition of the present invention contains: (A) a resin (hereinafter, also referred to as the “component (A)”); and (B) a solvent containing: (B1) a compound represented by the general formula (b-1) (hereinafter, also referred to as the “component (B)”). In the present invention, the “resist auxiliary film” refers to all the films used for the upper layer of a resist and films used for the underlayer of a resist, and examples thereof include a resist upper layer film, a resist intermediate layer film, and a resist underlayer film.

The resist auxiliary film composition of one aspect of the present invention preferably further contains: (C) at least one additive selected from the group consisting of a photosensitizer and an acid generating agent (hereinafter, also referred to as the “component (C)”).

Then, in the resist auxiliary film composition of the present invention, the content of the active component is limited to 45% by mass or less, based on the total amount (100% by mass) of the resist auxiliary film composition.

As used herein, the “active component” refers to the components excluding the component (B) among the components contained in the resist auxiliary film composition. Specifically, the active component encompasses the resin (A) and the additive (C), as well as an acid cross-linking agent, an acid diffusion controlling agent, a dissolution accelerator, a dissolution controlling agent, a sensitizing agent, a surfactant, an organic carboxylic acid or phosphorus oxoacid or a derivative thereof, a dye, a pigment, an adhesion aid, a halation preventing agent, a storage stabilizing agent, a defoaming agent, a shape improver, and the others that may be contained as other additives as described below.

Typically, for example, a thick resist auxiliary film is required to be formed to use the film as an etching mask. However, when a resist auxiliary film composition having a low resin content is used, it is difficult to form a thick resist auxiliary film.

In contrast, the resist auxiliary film composition of the present invention can be a photoresist auxiliary film material capable of forming a thick resist auxiliary film owing to use of the compound represented by the general formula (b-1) as the solvent, in spite of a reduced content of the active component including the resin of 45% by mass or less. In addition, since the content of the active component is reduced to 45% by mass or less, the resist auxiliary film composition of the present invention has an economical advantage.

In the resist auxiliary film composition of one aspect of the present invention, the content of the active component may be appropriately set depending on the application, and may be 42% by mass or less, 40% by mass or less, 36% by mass or less, 31% by mass or less, 26% by mass or less, 23% by mass or less, 20% by mass or less, 18% by mass or less, 16% by mass or less, 12% by mass or less, 10% by mass or less, 6% by mass or less, or 3% by mass or less, based on the total amount (100% by mass) of the resist auxiliary film composition.

On the other hand, the lower limit of the content of the active component is appropriately set depending on the application, and the content may be 1% by mass or more, 2% by mass or more, 4% by mass or more, 7% by mass or more, or 10% by mass or more, based on the total amount (100% by mass) of the resist auxiliary film composition.

The range of the content of the active component can be specified by any combination of an upper limit value and a lower limit value appropriately selected from the options each mentioned above.

In the resist auxiliary film composition of one aspect of the present invention, the content of the component (A) in the active component is preferably 50 to 100% by mass, more preferably 60 to 100% by mass, further preferably 70 to 100% by mass, further more preferably 75 to 100% by mass, and particularly preferably 80 to 100% by mass, based on the total amount (100% by mass) of the active component contained in the resist auxiliary film composition, in view of producing a photoresist auxiliary film material capable of forming a thick resist auxiliary film.

The resist auxiliary film composition of one aspect of the present invention may contain other components in addition to the above components (A) to (C) depending on the application.

However, in the resist auxiliary film composition of one aspect of the present invention, the total content of the components (A), (B), and (C) is preferably 30 to 100% by mass, more preferably 40 to 100% by mass, further preferably 60 to 100% by mass, further more preferably 80 to 100% by mass, and particularly preferably 90 to 100% by mass, based on the total amount (100% by mass) of the resist auxiliary film composition.

Hereinafter, details of each component contained in the resist auxiliary film composition of one aspect of the present invention will be described.

### <Component (A): Resin>

The resin (A) contained in the resist auxiliary film composition of one aspect of the present invention is not particularly limited. A known resin can be used, including a resin for a material for an antireflection film for KrF excimer laser or ArF excimer laser, or for a photoresist underlayer film for EUV lithography; a high carbon concentration resin for a spin-on-carbon film used in a two-layer resist method or a three-layer resist method; a silicon-containing resin for a spin-on-glass film used in a two-layer resist method or a three-layer resist method; or furthermore, a resin for an upper layer film of a photoresist that is intended to prevent pollution, cut light of unnecessary wavelength, or waterproof to cope with liquid immersion exposure, and an appropriate resin is selected depending on the application. As used herein, the “resin” encompasses a polymer having a predetermined constitutional unit, and also a compound having a predetermined structure.

The weight average molecular weight (Mw) of the resin used in one aspect of the present invention is preferably 500 to 50,000, more preferably 1,000 to 40,000, and further preferably 1,000 to 30,000.

In the resist auxiliary film composition of the present invention, the content of the component (A) may be appropriately set depending on the application, and may be 45% by mass or less, 42% by mass or less, 40% by mass or less, 35% by mass or less, 31% by mass or less, 26% by mass or less, 23% by mass or less, 20% by mass or less, 18% by mass or less, 16% by mass or less, 12% by mass or less, 10% by mass or less, 6% by mass or less, or 3% by mass or less, based on the total amount (100% by mass) of the resist auxiliary film composition.

The lower limit of the content of the component (A) is also appropriately set depending on the application, and the content may be 1% by mass or more, 2% by mass or more, 4% by mass or more, 7% by mass or more, or 10% by mass or more, based on the total amount (100% by mass) of the resist auxiliary film composition.

The range of the content of the component (A) can be specified by any combination of an upper limit value and a lower limit value appropriately selected from the options each mentioned above.

The resist auxiliary film composition is suitably used as a material for the antireflection film for KrF excimer laser or ArF excimer laser, or the photoresist underlayer film for EUV lithography, the spin-on-carbon film used in a two-layer resist method or a three-layer resist method, and the spin-on-glass film used in a three-layer resist method.

For example, in the case of use as a material for the antireflection film for KrF excimer laser or ArF excimer laser or the photoresist underlayer film for EUV lithography, the resin (A) desirably contains a novolac resin (A1) or an ethylenically unsaturated resin (A2).

In the case of use for the spin-on-carbon film used in a two-layer resist method or a three-layer resist method, the resin (A) desirably contains a high carbon resin (A3). In the case of use for the spin-on-glass film used in a three-layer resist method, the resin (A) desirably contains a silicon-containing resin (A4).

The resin (A) contained in the resist auxiliary film composition of one aspect of the present invention may contain only one selected from the group consisting of these resins (A1), (A2), (A3), and (A4), or may contain two or more thereof in combination.

As the resin (A), a resin other than the resins (A1), (A2), (A3), or (A4) may be contained.

However, the total content of the resins (A1), (A2), (A3), and (A4) in the resin (A) used in one aspect of the present invention is preferably 60 to 100% by mass, more preferably 70 to 100% by mass, further preferably 80 to 100% by mass, further more preferably 90 to 100% by mass, and particularly preferably 95 to 100% by mass, based on the total amount (100% by mass) of the resin (A).

Hereinafter, these resins (A1), (A2), (A3), and (A4) will be described.

### [Novolac Resin (A1)]

Examples of the novolac resin (A1) used in one aspect of the present invention include resins obtained by reacting a phenol with at least one of an aldehyde and a ketone in the presence of an acid catalyst (e.g., hydrochloric acid, sulfuric acid, and oxalic acid). The novolac resin (A1) is not particularly limited, and a known resin is used. For example, resins exemplified in Japanese Patent Laid-Open No. 2009-173623, International Publication No. WO 2013-024779, and International Publication No. WO 2015-137486 can be used.

Examples of the phenol include phenol, orthocresol, metacresol, paracresol, 2,3-dimethylphenol, 2,5-dimethylphenol, 3,4-dimethylphenol, 3,5-dimethylphenol, 2,4-dimethylphenol, 2,6-dimethylphenol, 2,3,5-trimethylphenol, 2,3,6-trimethylphenol, 2-t-butylphenol, 3-t-butylphenol, 4-t-butylphenol, 2-methylresorcinol, 4-methylresorcinol, 5-methylresorcinol, 4-t-butylcatechol, 2-methoxyphenol, 3-methoxyphenol, 2-propylphenol, 3-propylphenol, 4-propylphenol, 2-isopropylphenol, 2-methoxy-5-methylphenol, 2-t-butyl-5-methylphenol, thymol, isothymol, 4,4′-biphenol, 1-naphthol, 2-naphthol, hydroxyanthracene, hydroxypyrene, 2,6-dihydroxynaphthalene, and 2,6-dihydroxynaphthalene.

These phenols may be used singly or in combination of two or more thereof.

Examples of the aldehyde include formaldehyde, paraformaldehyde, trioxane, acetaldehyde, propionaldehyde, benzaldehyde, phenylacetaldehyde, α-phenylpropionaldehyde, β-phenylpropionaldehyde, benzaldehyde, 4-biphenylaldehyde, o-hydroxy benzaldehyde, m-hydroxy benzaldehyde, p-hydroxy benzaldehyde, o-chlorobenzaldehyde, m-chlorobenzaldehyde, p-chlorobenzaldehyde, o-methylbenzaldehyde, m-methylbenzaldehyde, p-methylbenzaldehyde, p-ethyl benzaldehyde, 3,4-dimethylbenzaldehyde, p-n-propylbenzaldehyde, p-n-butylbenzaldehyde, and terephthalaldehyde.

Examples of the ketone include acetone, methyl ethyl ketone, diethyl ketone, acetophenone, and diphenyl ketone.

These aldehydes and ketones may be used singly or in combination of two or more thereof.

Among these, the novolac resin (A1) used in one aspect of the present invention is preferably a resin obtained by a condensation reaction of cresol with an aldehyde, more preferably a resin obtained by a condensation reaction of at least one of metacresol and paracresol with at least one of formaldehyde and paraformaldehyde, and further preferably a resin obtained by a condensation reaction of combination of metacresol and paracresol with at least one of formaldehyde and paraformaldehyde.

When metacresol and paracresol are used in combination, the blending ratio by mass of metacresol to paracresol [metacresol/paracresol] as starting materials is preferably 10/90 to 90/10, more preferably 20/80 to 80/20, and further preferably 50/50 to 70/30.

As the novolac resin (A1) used in one aspect of the present invention, commercial products such as “EP4080G” and “EP4050G” (both manufactured by ASAHI YUKIZAI CORPORATION, cresol novolac resin) may be used.

The weight average molecular weight (Mw) of the novolac resin (A1) used in one aspect of the present invention is preferably 500 to 30,000, more preferably 1,000 to 20,000, further preferably 1,000 to 15,000, and further more preferably 1,000 to 10,000.

### [Ethylenically Unsaturated Resin (A2)]

The ethylenically unsaturated resin (A2) used in one aspect of the present invention is not particularly limited, and a known resin is used. The ethylenically unsaturated resin (A2) may be (A2a) a resin having at least one of: (a2-1) a constitutional unit derived from a phenolic hydroxyl group-containing compound and (a2-2) a constitutional unit capable of being decomposed by an action of an acid, a base, or heat to form an acid functional group, or may be a copolymer having both the constitutional unit (a2-1) and the constitutional unit (a2-2).

The solubility in the compound (B1) can be increased by using the resin having at least one of the constitutional unit (a2-1) and the constitutional unit (a2-2).

In the resin (A2a) used in one aspect of the present invention, the total content of the constitutional unit (a2-1) and the constitutional unit (a2-2) is preferably 30 mol % or more, more preferably 50 mol % or more, further preferably 60 mol % or more, further more preferably 70 mol % or more, and particularly preferably 80 mol % or more, based on the total amount (100 mol %) of the constitutional unit of the resin (A2a).

When the resin (A2a) used in one aspect of the present invention is the copolymer having both the constitutional unit (a2-1) and the constitutional unit (a2-2), the content ratio of the constitutional unit (a2-1) to the constitutional unit (a2-2) [(a2-1)/(a2-2)] is preferably 1/10 to 10/1, more preferably 1/5 to 8/1, further preferably 1/2 to 6/1, and further more preferably 1/1 to 4/1 in a molar ratio.

Examples of the phenolic hydroxyl group-containing compound for the constitutional unit (a2-1) include hydroxystyrene (o-hydroxystyrene, m-hydroxystyrene, and p-hydroxystyrene) and isopropenylphenol (o-isopropenylphenol, m-isopropenylphenol, and p-isopropenylphenol), and hydroxystyrene is preferable.

Examples of the acid functional group that may be formed from the constitutional unit (a2-2) when the unit is decomposed by the action of an acid, a base, or heat include a phenolic hydroxyl group and a carboxyl group.

Examples of the monomer for the constitutional unit capable of forming a phenolic hydroxyl group include hydroxy (α-methyl) styrenes protected by an acetal group, such as p-(1-methoxyethoxy) styrene, p-(1-ethoxyethoxy) styrene, p-(1-n-propoxyethoxy) styrene, p-(1-i-propoxyethoxy) styrene, p-(1-cyclohexyloxyethoxy) styrene, and α-methyl substituents thereof: p-acetoxystyrene, t-butoxycarbonylstyrene, t-butoxystyrene, and α-methyl substituents thereof.

These may be used singly or in combination of two or more thereof.

Examples of the monomer for the constitutional unit capable of forming a carboxyl group include (meth)acrylates protected by an acid decomposable ester group such as t-butyl (meth)acrylate, tetrahydropyranyl (meth)acrylate, 2-methoxybutyl (meth)acrylate, 2-ethoxyethyl (meth)acrylate, 2-t-butoxycarbonylethyl (meth)acrylate, 2-benzyloxycarbonylethyl (meth)acrylate, 2-phenoxycarbonylethyl (meth)acrylate, 2-cyclohexyloxycarbonyl (meth)acrylate, 2-isobornyloxycarbonylethyl (meth)acrylate, and 2-tricyclodecarbonyloxycarbonylethyl (meth)acrylate.

These may be used singly or in combination of two or more thereof.

Among these, as the monomer for the constitutional unit (a2-2), at least one selected from the group consisting of t-butyl (meth)acrylate, tetrahydropyranyl (meth)acrylate, 2-cyclohexyloxycarbonylethyl (meth)acrylate, and p-(1-ethoxyethoxy) styrene is preferable.

The resin (A2a) used in one aspect of the present invention, which is a resin having at least one of the constitutional unit (a2-1) and the constitutional unit (a2-2) as described above, may also have other constitutional units different from these constitutional units.

Examples of the monomer for such other constitutional units include alkyl (meth)acrylates: hydroxy group-containing monomers: epoxy group-containing monomers: cycloaliphatic structure-containing monomers: olefins such as ethylene, propylene, and isobutylene: halogenated olefins such as vinyl chloride and vinylidene chloride; diene monomers such as butadiene, isoprene, and chloroprene: aromatic vinyl monomers such as styrene, α-methylstyrene, p-methylstyrene, p-chlorostyrene, and p-methoxystyrene; cyano group-containing vinyl monomers such as (meth)acrylonitrile and cyanated vinylidene; (meth)acrylamides such as (meth)acrylamide, N,N-dimethyl (meth)acrylamide, N,N-dimethylol (meth)acrylamide; and heteroatom-containing cycloaliphatic vinyl monomers such as (meth)acryloylmorpholine, N-vinyl pyrrolidone, and N-vinyl caprolactam.

Examples of the alkyl (meth)acrylate include compounds other than the monomer for the constitutional unit (a2-2), such as methyl (meth)acrylate, ethyl (meth)acrylate, and propyl (meth)acrylate (n-propyl (meth)acrylate and i-propyl (meth)acrylate).

Examples of the hydroxy group-containing monomer include hydroxyalkyl (meth)acrylates such as 2-hydroxyethyl (meth)acrylate, 2-hydroxypropyl (meth)acrylate, 3-hydroxypropyl (meth)acrylate, 2-hydroxy butyl (meth)acrylate, 3-hydroxy butyl (meth)acrylate, and 4-hydroxy butyl (meth)acrylate.

The number of carbon atoms of the alkyl group contained in the hydroxyalkyl (meth)acrylate is preferably 1 to 10, more preferably 1 to 8, further preferably 1 to 6, and further more preferably 2 to 4, and the alkyl group may be a linear alkyl group or a branched alkyl group.

Examples of the epoxy-containing monomer include epoxy group-containing (meth)acrylates such as glycidyl (meth)acrylate, β-methylglycidyl (meth)acrylate, (3,4-epoxy cyclohexyl)methyl (meth)acrylate, and 3-epoxycyclo-2-hydroxypropyl (meth)acrylate: glycidyl crotonate, and allyl glycidyl ether.

Examples of the cycloaliphatic structure-containing monomer include cycloalkyl (meth)acrylates such as cyclopropyl (meth)acrylate, cyclobutyl (meth)acrylate, cyclopentyl (meth)acrylate, cyclohexyl (meth)acrylate, cycloheptyl (meth)acrylate, and cyclooctyl (meth)acrylate, isobornyl (meth)acrylate, and dicyclopentenyl (meth)acrylate.

As the resin (A2a) used in one aspect of the present invention, a resin having a constitutional unit derived from adamantyl (meth)acrylate as the constitutional unit derived from the cycloaliphatic structure-containing monomer may be used. Such a resin corresponds to not only the resin (A2a), but also the resin (A2b) described below.

The resin (A2a) used in one aspect of the present invention may have a constitutional unit derived from a monomer selected from the group consisting of an ester of a compound having two or more hydroxyl groups in the molecule, such as a dihydric or higher polyhydric alcohol, polyether diol, and polyester diol with a (meth)acrylic acid: an adduct of a compound having two or more epoxy groups in the molecule, exemplified by an epoxy resin, with a (meth)acrylic acid; and a condensate of a compound having two or more amino groups in the molecule with a (meth)acrylic acid.

Examples of such a monomer include (poly)alkylene glycol (derivative) di(meth)acrylates such as ethylene glycol di(meth)acrylate, diethylene glycol di(meth)acrylate, triethylene glycol di(meth)acrylate, propylene glycol di(meth)acrylate, dipropylene glycol di(meth)acrylate, tripropylene glycol di(meth)acrylate, butanediol di(meth)acrylate, trimethylolpropane di(meth)acrylate, trimethylolpropane tri(meth)acrylate, pentaerythritol tri(meth)acrylate, pentaerythritol tetra(meth)acrylate, tricyclodecane dimethanol di(meth)acrylate, trimethylolpropane tri(meth)acrylate, N,N′-methylenebis(meth)acrylamide, and di(meth)acrylate of an ethylene glycol adduct or a propylglycol adduct of bisphenol A; and epoxy (meth)acrylates such as a (meth)acrylic acid adduct of bisphenol A diglycidyl ether.

The weight average molecular weight (Mw) of the resin (A2a) used in one aspect of the present invention is preferably 400 to 50,000, more preferably 1,000 to 40,000, further preferably 1,000 to 30,000, and further more preferably 1,000 to 25,000.

The resin (A2) used in one aspect of the present invention may be (A2b) a resin having: (b2-1) a constitutional unit having an adamantane structure, and desirably has a constitutional unit capable being decomposed by an action of an acid to forming an acid functional group. In view of the solubility in a solvent and the adhesiveness to a substrate, the resin (A2) used in one aspect of the present invention is practically preferably a copolymer having: (b2-2) a constitutional unit having a lactone structure, together with the constitutional unit (b2-1).

At least one of the hydrogen atoms to which carbon atoms constituting the adamantane structure in the constitutional unit (b2-1) are bonded may be replaced with a substituent R.

Also, at least one of the hydrogen atoms to which carbon atoms constituting the lactone structure in the constitutional unit (b2-2) are bonded may be replaced with the substituent R.

Examples of the substituent R include an alkyl group having 1 to 6 carbon atoms, a hydroxyalkyl group having 1 to 6 carbon atoms, a cycloalkyl group having 3 to 6 carbon atoms, a halogen atom (fluorine atom, chlorine atom, bromine atom, and iodine atom), a deuterium atom, a hydroxy group, an amino group, a nitro group, a cyano group, and a group represented by the following formula (i) or (ii).

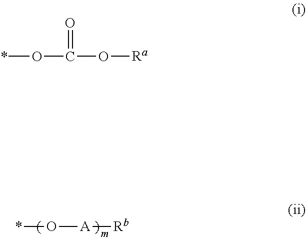

In the above formula (i) or (ii), Ra and Rb are each independently an alkyl group having 1 to 6 carbon atoms, a hydroxyalkyl group having 1 to 6 carbon atoms, or a cycloalkyl group having 3 to 6 carbon atoms.

m is an integer of 1 to 10, preferably an integer of 1 to 6, more preferably an integer of 1 to 3, and further preferably an integer of 1 to 2.

A is an alkylene group having 1 to 6 carbon atoms (preferably 1 to 4 carbon atoms, and more preferably 2 to 3).

Examples of the alkylene group include a methylene group, an ethylene group, an n-propylene group, an i-propylene group, a 1,4-butylene group, a 1,3-butylene group, a tetramethylene group, a 1,5-pentylene group, a 1,4-pentylene group, and a 1,3-pentylene group.

The resin (A2b) used in one aspect of the present invention may have: (b2-1a) the constitutional unit having an adamantane structure substituted with a hydroxy group, which is a constitutional unit (b2-1), and in the resin (A2b), the content of the constitutional unit (b2-1a) is preferably less than 50 mol %, more preferably less than 44 mol %, further preferably less than 39 mol %, and further more preferably less than 34 mol %, based on the total amount (100 mol %) of the constitutional unit of the resin (A2b).

In one aspect of the present invention, the constitutional unit (b2-1) is preferably a constitutional unit (b2-1-1) represented by the following formula (b2-1-i) or a constitutional unit (b2-1-2) represented by the following formula (b2-1-ii).

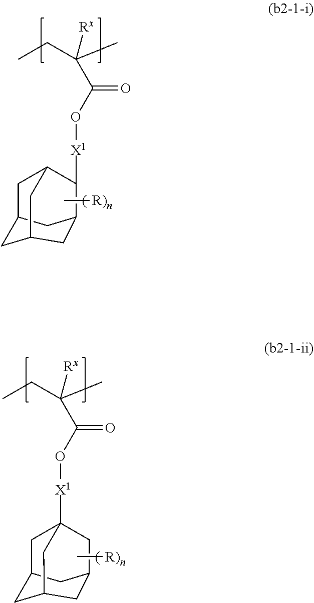

In the above formula, n is each independently an integer of 0 to 14, preferably an integer of 0 to 4, more preferably an integer of 0 to 2, and further preferably an integer of 0 to 1.

R^{x} is each independently a hydrogen atom or a methyl group.

R is each independently a substituent R that may be included in the adamantane structure, and is specifically as described above. R is preferably an alkyl group having 1 to 6 carbon atoms, and more preferably an alkyl group having 1 to 3 carbon atoms.

X¹ is each independently a single bond, an alkylene group having 1 to 6 carbon atoms, or a divalent linking group represented by any of the following formulas.

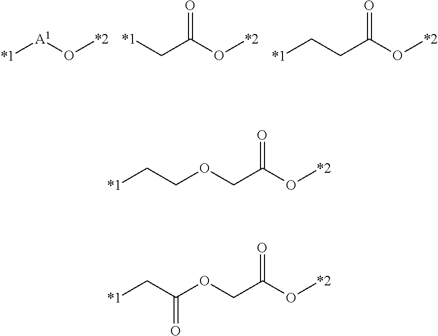

In the above formula, *1 represents a binding site with an oxygen atom in the above formula (b2-1-i) or (b2-1-ii), *2 represents a binding site with a carbon atom in the adamantane structure. A¹ represents an alkylene group having 1 to 6 carbon atoms.

In one aspect of the present invention, the constitutional unit (b2-2) is preferably any of a constitutional unit (b2-2-1) represented by the following formula (b2-2-i), a constitutional unit (b2-2-2) represented by the following formula (b2-2-ii), and a constitutional unit (b2-2-3) represented by the following formula (b2-2-iii).

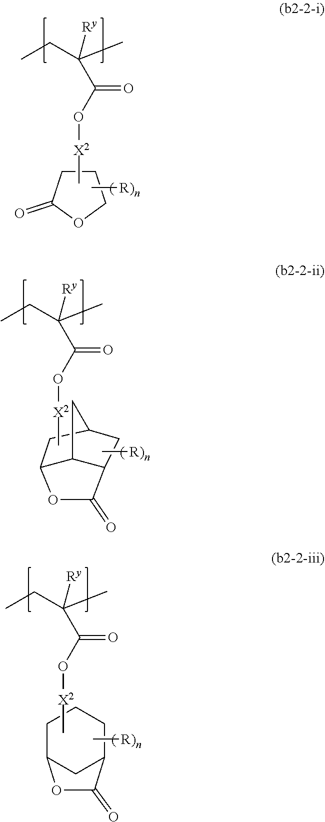

In the above formula, n1 is an integer of 0 to 5, preferably an integer of 0 to 2, and more preferably an integer of 0 to 1.
n2 is an integer of 0 to 9, preferably an integer of 0 to 2, and more preferably an integer of 0 to 1.
n3 is an integer of 0 to 9, preferably an integer of 0 to 2, and more preferably an integer of 0 to 1.
R^{y} is a hydrogen atom or a methyl group.
R is each independently a substituent R that may be included in the lactone structure, and is specifically as described above. R is preferably an alkyl group having 1 to 6 carbon atoms, and more preferably an alkyl group having 1 to 3 carbon atoms. When a plurality of R is present, the plurality of R may be the same groups or groups different from each other.
X² is a single bond, an alkylene group having 1 to 6 carbon atoms, or a divalent linking group represented by any of the following formulas.

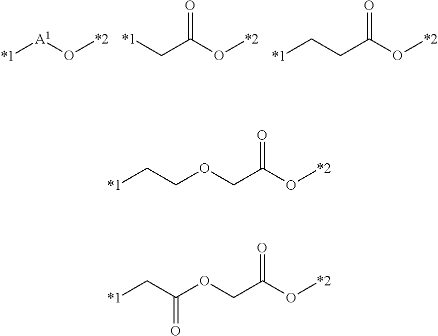

In the above formula, *1 represents a binding site with an oxygen atom in the above formula (b2-2-i), (b2-2-ii), or (b2-2-iii), and *2 represents a binding site with a carbon atom in the lactone structure. A¹ represents an alkylene group having 1 to 6 carbon atoms.

The resin (A²b) used in one aspect of the present invention may have other constitutional units in addition to the constitutional units (b2-1) and (b2-2).

Examples of such other constitutional units include constitutional units derived from monomers such as alkyl (meth)acrylate; hydroxy group-containing monomer; epoxy group-containing monomer; a cycloaliphatic structure-containing monomer; an olefin such as ethylene, propylene, and isobutylene; halogenated olefins such as vinyl chloride and vinylidene chloride; diene monomers such as butadiene, isoprene, and chloroprene; styrene, α-methylstyrene, vinyl toluene, acrylonitrile, (meth)acrylamide, (meth)acrylonitrile, (meth)acryloylmorpholine, and N-vinyl pyrrolidone. The details of these monomers are the same as the description in the item of the resin (A2a).

In the resin (A2b) used in one aspect of the present invention, the total content of the constitutional units (b2-1) and (b2-2) is preferably 30 to 100 mol %, more preferably 50 to 100 mol %, further preferably 70 to 100 mol %, further more preferably 80 to 100 mol %, and particularly preferably 90 to 100 mol %, based on the total amount (100 mol %) of the constitutional unit of the resin (A2b).

The weight average molecular weight (Mw) of the resin (A2b) used in one aspect of the present invention is preferably 400 to 50,000, more preferably 2,000 to 40,000, further preferably 3,000 to 30,000, and further more preferably 4,000 to 20,000.

The molecular weight distribution (Mw/Mn) of the resin (A2b) is preferably 6.0 or less, more preferably 5.0 or less, further preferably 4.0 or less, and further more preferably 3.2 or less, and preferably 1.01 or more, more preferably 1.05 or more, and further preferably 1.1 or more.

The resin (A2) used in one aspect of the present invention may be (A2c) a resin having any two or more constitutional units of: (a2-1) the constitutional unit derived from a phenolic hydroxyl group-containing compound, (a2-2) the constitutional unit capable of being decomposed by an action of an acid, a base, or heat to form an acid functional group, (b2-1) the constitutional unit having an adamantane structure, and (b2-2) the constitutional unit having a lactone structure (provided that the resin (A2a) and the resin (A2b) are excluded). The resin (A2c) is not particularly limited, and a known resin may be used. Example thereof that can be used include resins exemplified in a book “40 years of lithography technology”, International Publication No. WO 2014-175275, International Publication No. WO 2015-115613, International Publication No. WO 2020-137935, International Publication No. WO 2021-029395, and International Publication No. WO 2021-029396.

The weight average molecular weight (Mw) of the resin (A2c) used in one aspect of the present invention is preferably 500 to 50,000, more preferably 2,000 to 40,000, further preferably 3,000 to 30,000, and further more preferably 4,000 to 20,000.

The molecular weight distribution (Mw/Mn) of the resin (A2c) is preferably 6.0 or less, more preferably 5.0 or less, further preferably 4.0 or less, and further more preferably 3.2 or less, and preferably 1.01 or more, more preferably 1.05 or more, and further preferably 1.1 or more.

### [High Carbon Resin (A3)]

The high carbon resin (A3) used in one aspect of the present invention refers to a resin in which the weight of the carbon atoms contained exceeds 60% of the weight of the whole elements. Above all, a resin in which the weight of the carbon atoms exceeds 70% is preferable, a resin in which the weight of the carbon atoms exceeds 80% is more preferable, and a resin in which the weight of the carbon atoms exceeds 90% is further more preferable. Specific examples of the high carbon resin (A3) include, but are not particularly limited to, known resins described in International Publication No. WO 2020/145406.

The weight average molecular weight (Mw) of the resin (A3) used in one aspect of the present invention is preferably 400 to 50,000, more preferably 2,000 to 40,000, further preferably 3,000 to 30,000, and further more preferably 4,000 to 20,000.

The molecular weight distribution (Mw/Mn) of the resin (A3) is preferably 6.0 or less, more preferably 5.0 or less, further preferably 4.0 or less, and further more preferably 3.2 or less, and preferably 1.01 or more, more preferably 1.05 or more, and further preferably 1.1 or more.

### [Silicon-Containing Resin (A4)]

The silicon-containing resin (A4) used in one aspect of the present invention is not particularly limited as long as it is a resin containing a silicon atom, and examples thereof include known resins described in Japanese Patent Laid-Open No. 2007-226170 and Japanese Patent Laid-Open No. 2007-226204.

The weight average molecular weight (Mw) of the resin (A4) used in one aspect of the present invention is preferably 400 to 50,000, more preferably 2,000 to 40,000, further preferably 3,000 to 30,000, and further more preferably 4,000 to 20,000.

The molecular weight distribution (Mw/Mn) of the resin (A4) is preferably 6.0 or less, more preferably 5.0 or less, further preferably 4.0 or less, and further more preferably 3.2 or less, and preferably 1.01 or more, more preferably 1.05 or more, and further preferably 1.1 or more.

### <Component (B): Solvent>

The resist auxiliary film composition of one aspect of the present invention contains: (B) a solvent containing (B1) a compound represented by the following general formula (b-1).

The compound (B1) may be used singly or in combination of two or more thereof.

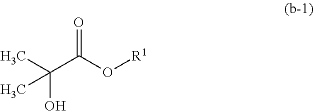

In the above formula (b-1), R¹ is an alkyl group having 1 to 10 carbon atoms. The alkyl group may be a linear alkyl group or a branched alkyl group.

Examples of the alkyl group capable of being selected as R¹ include a methyl group, an ethyl group, an n-propyl group, an i-propyl group, an n-butyl group, an i-butyl group, an s-butyl group, or a t-butyl group, a pentyl group, a hexyl group, a heptyl group, an octyl group, a 2-ethylhexyl group, a nonyl group, and a decyl group.

Among these, R¹ in the general formula (b-1) is preferably a methyl group, an ethyl group, an n-propyl group, an i-propyl group, an n-butyl group, an i-butyl group, an s-butyl group, or a t-butyl group, in one aspect of the present invention, more preferably an ethyl group, an n-propyl group, an i-propyl group, an n-butyl group, an i-butyl group, an s-butyl group, or a t-butyl group, further preferably an n-propyl group, an i-propyl group, an n-butyl group, an i-butyl group, an s-butyl group, or a t-butyl group, and further more preferably an i-propyl group, an n-butyl group, or an i-butyl group.

In the resist auxiliary film composition of one aspect of the present invention, (B2) a solvent other than the compound (B1) may be contained as the component (B).

Examples of the solvent (B2) include lactones such as γ-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond such as ethylene glycol monoacetate, diethylene glycol monoacetate, propylene glycol monoacetate, and dipropylene glycol monoacetate; compounds having an ether bond, such as mono alkyl ethers such as monomethyl ether, monoethyl ether, monopropyl ether, and monobutyl ether or monophenyl ethers of the polyhydric alcohol or the compounds having an ester bond such as 1-methoxy 2-propanol; cyclic ethers such as dioxane, and esters other than the compound (B1), such as methyl lactate, ethyl lactate, methyl acetate, ethyl acetate, butyl acetate, methyl pyruvate, ethyl pyruvate, methyl α-methoxyisobutyrate, methyl β-methoxyisobutyrate, ethyl 2-ethoxyisobutyrate, methyl methoxypropionate, ethyl ethoxypropionate, methyl α-formyloxyisobutyrate, methyl β-formyl oxyisobutyrate, and methyl 3-hydroxyisobutyrate; aromatic organic solvents such as anisole, ethylbenzyl ether, cresyl methyl ether, diphenyl ether, dibenzyl ether, phenetole, butyl phenyl ether, ethylbenzene, diethylbenzene, pentylbenzene, isopropylbenzene, toluene, xylene, cymene, and mesitylene; and dimethylsulfoxide (DMSO).

These solvents (B2) may be used singly or in combination of two or more thereof.

However, in the resist auxiliary film composition of the present invention, the content of the compound (B1) in the component (B) is preferably 20 to 100% by mass, more preferably 30 to 100% by mass, further preferably 50 to 100% by mass, further more preferably 60 to 100% by mass, and particularly preferably 70 to 100% by mass, based on the total amount (100% by mass) of the component (B) contained in the resist auxiliary film composition, in view of producing a photoresist auxiliary film material capable of forming a thick resist auxiliary film.

The component (B) used in one aspect of the present invention preferably contains one or more selected from the group consisting of methyl α-methoxyisobutyrate, methyl α-formyloxyisobutyrate, methyl α-acetyloxyisobutyrate, methyl 3-hydroxyisobutyrate, and 1-methoxy 2-propanol as the solvent (B2), in view of the solubility of the acid generating agent used in the resist auxiliary film composition. It is preferable to contain methyl α-methoxyisobutyrate, in view of the solubility of the resin used in the resist auxiliary film composition. It is preferable to contain methyl α-formyloxyisobutyrate or methyl α-acetyloxyisobutyrate, in view of forming a thick soluble resist film from the resin used in the resist auxiliary film composition. It is preferable to contain methyl 3-hydroxyisobutyrate, in view of obtaining a coating film having a surface in a good condition by high temperature baking. It is preferable to contain 1-methoxy-2-propanol, in view of obtaining a coating film having high in-plane uniformity.

The method for mixing methyl α-methoxyisobutyrate, methyl α-formyloxyisobutyrate, methyl α-acetyloxyisobutyrate, methyl 3-hydroxyisobutyrate, or 1-methoxy-2-propanol is not particularly limited, and they can be contained by either a method including adding methyl α-methoxyisobutyrate, methyl α-formyloxyisobutyrate, methyl α-acetyloxyisobutyrate, methyl 3-hydroxyisobutyrate, or 1-methoxy-2-propanol to the compound (B1), or a method including mixing the component (B) by forming any of them as a by-product or incorporating any of them in the manufacturing process of the compound (B1).

The content of the solvent (B2) is not limited, and is preferably less than 100% by mass based on the total amount (100% by mass) of the compound (B1), in view of shortening the drying time of the coating film to improve productivity. The content is preferably 70% by mass or less, and more preferably 60% by mass or less, 50% by mass or less, 40% by mass or less, 30% by mass or less, 20% by mass or less, 10% by mass or less, 5% by mass or less, 1% by mass or less, in view of increasing the solvency of the solvent while ensuring a moderate drying time, and is further preferably 0.1% by mass or less, and particularly preferably 0.01% by mass or less. The content of the solvent (B2) is preferably 0.0001% by mass or more in view of improving the storage stability of the resist auxiliary film composition, more preferably 0.001% by mass or more in view of improving the solubility of the active component of the resist auxiliary film composition, and further preferably 0.01% by mass or more in view of suppressing the defect of the resist auxiliary film.

The content of methyl α-methoxyisobutyrate, methyl α-formyloxyisobutyrate, methyl α-acetyloxyisobutyrate, methyl 3-hydroxyisobutyrate, or 1-methoxy-2-propanol is not limited, and is preferably less than 100% by mass, more preferably 70% by mass or less, 60% by mass or less, 50% by mass or less, 40% by mass or less, 30% by mass or less, 20% by mass or less, 10% by mass or less, 5% by mass or less, and 1% by mass or less, further preferably 0.1% by mass or less, and particularly preferably 0.01% by mass or less, based on the total amount (100% by mass) of the resist auxiliary film composition, in view of shortening the drying time of the coating film to improve productivity. The content thereof is preferably 0.0001% by mass or more in view of improving the storage stability of the resist auxiliary film composition, more preferably 0.001% by mass or more in view of improving the solubility of the active component of the resist auxiliary film composition, and further preferably 0.01% by mass or more in view of suppressing the defect of the resist auxiliary film.

The content of methyl α-methoxyisobutyrate, methyl α-formyloxyisobutyrate, methyl α-acetyloxyisobutyrate, methyl 3-hydroxyisobutyrate, or 1-methoxy-2-propanol is preferably 100% by mass or less, more preferably 70% by mass or less, 60% by mass or less, 50% by mass or less, 40% by mass or less, 30% by mass or less, 20% by mass or less, 10% by mass or less, 5% by mass or less, and 1% by mass or less, further preferably 0.1% by mass or less, and particularly preferably 0.01% by mass or less, based on the total amount (100% by mass) of the compound (B1), in view of improving productivity by shortening the drying time of the resist auxiliary film composition. The content thereof is preferably 0.0001% by mass or more in view of improving the storage stability of the resist auxiliary film composition, more preferably 0.001% by mass or more in view of improving the solubility of the active component of the resist auxiliary film composition, and further preferably 0.01% by mass or more in view of suppressing the defect of the resist auxiliary film.

The content of 1-methoxy-2-propanol is preferably 1 to 98% by mass, and more preferably 16 to 98% by mass, based on the total amount (100% by mass) of the resist auxiliary film composition, in view of the in-plane uniformity of the coating film. In addition, the content of 1-methoxy-2-propanol is also preferably 1 to 99% by mass, and also more preferably 30 to 99% by mass, based on the total amount (100% by mass) of the compound (B1).

The component (B) used in one aspect of the present invention preferably contains one or more selected from the group consisting of methyl α-formyloxyisobutyrate, methyl α-acetyloxyisobutyrate, and methyl 3-hydroxyisobutyrate, as the solvent (B2).

In the resist auxiliary film composition of the present invention, the content of the component (B) may be appropriately set depending on the application, and may be 50% by mass or more, 54% by mass or more, 58% by mass or more, 60% by mass or more, 65% by mass or more, 69% by mass or more, 74% by mass or more, 77% by mass or more, 80% by mass or more, 82% by mass or more, 84% by mass or more, 88% by mass or more, 90% by mass or more, 94% by mass or more, or 97% by mass or more, based on the total amount (100% by mass) of the resist auxiliary film composition.

The upper limit value of the content of the component (B) may be appropriately set according to the content of the component (A), and the content may be 99% by mass or less, 98% by mass or less, 96% by mass or less, 93% by mass or less, 91% by mass or less, 86% by mass or less, 81% by mass or less, 76% by mass or less, 71% by mass or less, 66% by mass or less, or 61% by mass or less, based on the total amount (100% by mass) of the resist auxiliary film composition.

The range of the content of the component (B) can be specified by any combination of an upper limit value and a lower limit value appropriately selected from the options each mentioned above.

<Component (C): Additive Selected from Photosensitizer and Acid Generating Agent>

The resist auxiliary film composition of one aspect of the present invention preferably contains: (C) at least one additive selected from the group consisting of a photosensitizer and an acid generating agent.

The component (C) may be used singly or in combination of two or more thereof.

In the resist auxiliary film composition of one aspect of the present invention, the content of the component (C) is preferably 0.01 to 80 parts by mass, more preferably 0.05 to 65 parts by mass, further preferably 0.1 to 50 parts by mass, and further more preferably 0.5 to 30 parts by mass per 100 parts by mass of the resin (A) contained in the resist auxiliary film composition.

Hereinafter, the photosensitizer and the acid generating agent contained as the component (C) will be described.

### [Photosensitizer]

The photosensitizer that may be selected as the component (C) is not particularly limited, as long as it is typically used as the photosensitive component in the resist auxiliary film composition. A photosensitizer used in a resist composition can also be used.

The photosensitizers may be used singly or in combination of two or more thereof.

Examples of the photosensitizer used in one aspect of the present invention include a reactant of acid chloride and a compound having a functional group condensable with the acid chloride (such as a hydroxyl group and an amino group).

Examples of the acid chloride include naphthoquinonediazidosulfonic acid chloride and benzoquinonediazidosulfonic acid chloride, and specific examples thereof include 1,2-naphthoquinonediazido-5-sulfonyl chloride and 1,2-naphthoquinonediazido-4-sulfonyl chloride.

Examples of the compound condensable having a functional group with the acid chloride include hydroxybenzophenones such as hydroquinone, resorcin, 2,4-dihydroxy benzophenone, 2,3,4-trihydroxy benzophenone, 2,4,6-trihydroxybenzophenone, 2,4,4′-trihydroxybenzophenone, 2,3,4,4′-tetrahydroxybenzophenone, 2,2′,4,4′-tetrahydroxybenzophenone, and 2,2′,3,4,6′-pentahydroxybenzophenone; hydroxyphenylalkanes such as bis(2,4-dihydroxyphenyl) methane, bis(2,3,4-trihydroxyphenyl) methane, and bis(2,4-dihydroxyphenyl)propane; and hydroxytriphenylmethanes such as 4,4′,3″,4″-tetrahydroxy-3,5,3′,5′-tetramethyltriphenylmethane, and 4,4′,2″,3″,4″-pentahydroxy-3,5,3′,5′-tetramethyltriphenylmethane.

As the photosensitizer used in one aspect of the present invention, commercial products such as “DTEP-350” (a diazonaphthoquinone photosensitizer manufactured by DAITO CHEMIX Co., Ltd.) may be used.

### [Acid Generating Agent]

The acid generating agent that may be selected as the component (c) may be a compound capable of directly or indirectly generating an acid by heating or by irradiation with radiation such as a visible light, an ultraviolet, an excimer laser, an electron beam, an extreme ultraviolet (EUV), an X-ray, and an ion beam.

Specifically, as the suitable acid generating agent, a compound represented by any of the following general formulas (c-1) to (c-8) is preferable.

(Compound Represented by General Formula (c-1))

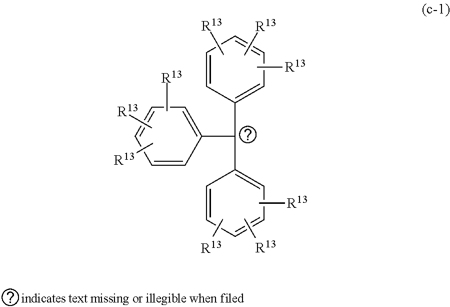

In the above formula (c-1), R¹³ is each independently a hydrogen atom, a linear, branched, or cyclic alkyl group, a linear, branched, or cyclic alkoxy group, a hydroxyl group, or a halogen atom.

X⁻ is a sulfonic acid ion or halide ion having an alkyl group, an aryl group, a halogen-substituted alkyl group, or a halogen-substituted aryl group.

The compound represented by the general formula (c-1) is preferably at least one selected from the group consisting of triphenylsulfonium trifluoromethanesulfonate, triphenylsulfonium nonafluoro-n-butanesulfonate, diphenyltolylsulfonium nonafluoro-n-butanesulfonate, triphenylsulfonium perfluoro-n-octanesulfonate, diphenyl-4-methylphenylsulfonium trifluoromethanesulfonate, di-2,4,6-trimethylphenylsulfonium trifluoromethanesulfonate, diphenyl-4-t-trifluoromethanesulfonate, diphenyl-4-t-butoxyphenylsulfonium nonafluoro-n-butanesulfonate, diphenyl-4-butoxyphenylsulfonium trifluoromethanesul fonate, bis(4-fluorophenyl)-4-hydroxy phenylsulfonium trifluoromethanesulfonate, diphenyl-4-hydroxyphenylsulfonium hydroxyphenylsulfonium nonafluoro-n-butanesulfonate, bis(4-hydroxyphenyl)-phenylsulfonium trifluoromethanesulfonate, tri (4-methoxyphenyl) sulfonium trifluoromethanesulfonate, tri (4-fluorophenyl) sulfonium trifluoromethanesulfonate, triphenylsulfonium p-toluene sulfonate, triphenylsulfonium benzenesulfonate, diphenyl-2,4,6-trimethylphenyl-p-toluene sulfonate, diphenyl-2,4,6-trimethylphenylsulfonium-2-trifluoromethylbenzenesulfonate, diphenyl-2,4,6-trimethylphenylsulfonium-4-trifluoromethylbenzenesulfonate, diphenyl-2,4,6-trimethylphenylsulfonium-2,4-difluorobenzenesulfonate, diphenyl-2,4,6-trimethylphenylsulfonium hexafluorobenzenesulfonate, diphenylnaphthylsulfonium trifluoromethanesulfonate, diphenyl-4-hydroxyphenylsulfonium-p-toluene sulfonate, triphenylsulfonium 10-camphorsulfonate, diphenyl-4-hydroxyphenylsulfonium 10-camphorsulfonate, and cyclo(1,3-perfluoropropanedisulfone) imidate.

(Compound Represented by General Formula (c-2))

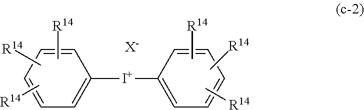

In the above formula (c-2), R¹⁴ is each independently a hydrogen atom, a linear, branched, or cyclic alkyl group, a linear, branched, or cyclic alkoxy group, a hydroxyl group, or a halogen atom.

X⁻ is a sulfonic acid ion or halide ion having an alkyl group, an aryl group, a halogen-substituted alkyl group, or a halogen-substituted aryl group.

The compound represented by the general formula (c-2) is preferably at least one selected from the group consisting of bis(4-t-butylphenyl) iodonium trifluoromethanesulfonate, bis(4-t-butylphenyl) iodonium nonafluoro-n-butanesulfonate, bis(4-t-butylphenyl) iodonium perfluoro-n-octanesulfonate, bis(4-t-butylphenyl) iodonium p-toluene sulfonate, bis(4-t-butylphenyl) iodonium benzenesulfonate, bis(4-t-butylphenyl) iodonium-2-trifluoromethylbenzenesulfonate, bis(4-t-butylphenyl) iodonium-4-trifluoromethylbenzenesulfonate, bis(4-t-butylphenyl) iodonium-2,4-difluorobenzenesulfonate, bis(4-t-butylphenyl) iodonium hexafluorobenzenesulfonate, bis(4-t-butylphenyl) iodonium 10-camphorsulfonate, diphenyliodonium trifluoromethanesulfonate, diphenyliodonium nonafluoro-n-butanesulfonate, diphenyliodonium perfluoro-n-octanesulfonate, diphenyliodonium p-toluene sulfonate, diphenyliodonium benzenesulfonate, diphenyliodonium 10-camphorsulfonate, diphenyliodonium-2-trifluoromethylbenzenesulfonate, diphenyliodonium-4-trifluoromethylbenzenesulfonate, diphenyliodonium-2,4-difluorobenzenesulfonate, diphenyliodonium hexafluorobenzenesulfonate, di(4-trifluoromethylphenyl) iodonium trifluoromethanesulfonate, di(4-trifluoromethylphenyl) iodonium nonafluoro-n-butanesulfonate, di(4-trifluoromethylphenyl) iodonium perfluoro-n-octanesulfonate, di(4-trifluoromethylphenyl) iodonium p-toluene sulfonate, di(4-trifluoromethylphenyl) iodonium benzenesulfonate, and di(4-trifluoromethylphenyl) iodonium 10-camphorsulfonate.

(Compound Represented by General Formula (c-3))

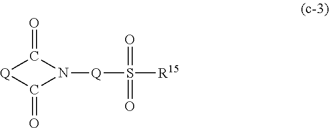

In the above formula (c-3), Q is an alkylene group, an arylene group, or an alkoxylene group. R¹⁵ is an alkyl group, an aryl group, a halogen-substituted alkyl group, or a halogen-substituted aryl group.

The compound represented by the general formula (c-3) is preferably at least one selected from the group consisting of N-(trifluoromethylsulfonyloxy) succinimide, N-(trifluoromethylsulfonyloxy)phthalimide, N-(trifluoromethylsulfonyloxy)diphenylmaleimide, N-(trifluoromethylsulfonyloxy)bicyclo[2.2.1]hept-5-ene-2,3-dicarboxyimido, N-(trifluoromethylsulfonyloxy)naphthylimido, N-(10-camphorsulfonyloxy)succinimide, N-(10-camphorsulfonyloxy)phthalimide, N-(10-camphorsulfonyloxy)diphenylmaleimide, N-(10-camphorsulfonyloxy)bicyclo[2.2.1]hept-5-ene-2,3-dicarboxyimido, N-(10-camphorsulfonyloxy)naphthylimido, N-(n-octanesulfonyloxy)bicyclo[2.2.1]hept-5-ene-2,3-dicarboxyimido, N-(n-octanesulfonyloxy)naphthylimido, N-(p-toluenesulfonyloxy)bicyclo[2.2.1]hept-5-ene-2,3-dicarboxyimido, N-(p-toluenesulfonyloxy)naphthylimido, N-(2-trifluoromethylbenzenesulfonyloxy)bicyclo[2.2.1]hept-5-ene-2,3-dicarboxyimido, N-(2-trifluoromethylbenzenesulfonyloxy)naphthylimido, N-(4-trifluoromethylbenzenesulfonyloxy)bicyclo[2.2.1]hept-5-ene-2,3-dicarboxyimido, N-(4-trifluoromethylbenzenesulfonyloxy)naphthylimido, N-(perfluorobenzenesulfonyloxy)bicyclo[2.2.1]hept-5-ene-2,3-dicarboxyimido, N-(perfluorobenzenesulfonyloxy)naphthylimido, N-(1-naphthalene sulfonyloxy)bicyclo[2.2.1]hept-5-ene-2,3-dicarboxyimido, N-(1-naphthalene sulfonyloxy)naphthylimido, N-(nonafluoro-n-butanesulfonyloxy)bicyclo[2.2.1]hept-5-ene-2,3-dicarboxyimido, N-(nonafluoro-n-butanesulfonyloxy)naphthylimido, N-(perfluoro-n-octanesulfonyloxy)bicyclo[2.2.1]hept-5-ene-2,3-dicarboxyimido, and N-(perfluoro-n-octanesulfonyloxy)naphthylimido.

(Compound Represented by General Formula (c-4))

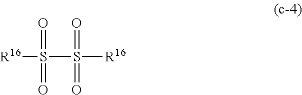

In the above formula (c-4), R¹⁶ is each independently a linear, branched, or cyclic alkyl group, an aryl group, a heteroaryl group, or an aralkyl group, and at least one hydrogen of these groups may be replaced with an arbitrary substituent.

The compound represented by the general formula (c-4) is preferably at least one selected from the group consisting of diphenyl disulfone, di(4-methylphenyl)disulfone, dinaphthyldisulfone, di(4-t-butylphenyl)disulfone, di(4-hydroxyphenyl)disulfone, di(3-hydroxynaphthyl)disulfone, di(4-fluorophenyl)disulfone, di(2-fluorophenyl)disulfone, and di(4-trifluoromethylphenyl)disulfone.

(Compound Represented by General Formula (c-5))

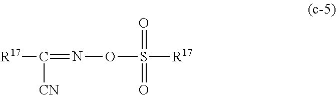

In the above formula (c-5), R¹⁷ is each independently a linear, branched, or cyclic alkyl group, an aryl group, a heteroaryl group, or an aralkyl group, and at least one hydrogen of these groups may be replaced with an arbitrary substituent.

The compound represented by the general formula (c-5) is preferably at least one selected from the group consisting of α-(methylsulfonyloxyimino)-phenylacetonitrile, α-(methylsulfonyloxyimino)-4-methoxyphenylacetonitrile, α-(trifluoromethylsulfonyloxyimino)-phenylacetonitrile, α-(trifluoromethylsulfonyloxyimino)-4-methoxyphenylacetonitrile, α-(ethylsulfonyloxyimino)-4-methoxyphenylacetonitrile, α-(propylsulfonyloxyimino)-4-methylphenylacetonitrile, and α-(methylsulfonyloxyimino)-4-bromophenylacetonitrile.

(Compound Represented by General Formula (c-6))

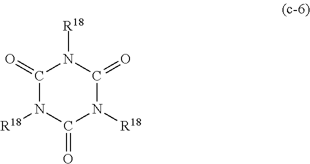

In the above formula (c-6), R¹⁸ is each independently a halogenated alkyl group having one or more chlorine atoms and one or more bromine atoms. The number of carbon atoms of the halogenated alkyl group is preferably 1 to 5.

(Compound Represented by General Formulas (c-7) and (c-8))

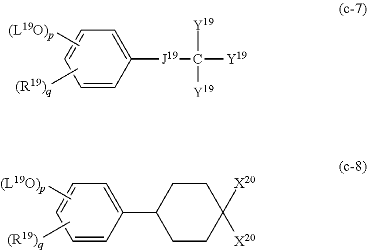

In the above formulas (c-7) and (c-8), R¹⁹ and R²⁰ are each independently an alkyl group having 1 to 3 carbon atoms (such as a methyl group, an ethyl group, an n-propyl group, or an i-propyl group), a cycloalkyl group having 3 to 6 carbon atoms (such as a cyclopentyl group or a cyclohexyl group), an alkoxyl group having 1 to 3 carbon atoms (such as a methoxy group, an ethoxy group, or a propoxy group), or an aryl group having 6 to 10 carbon atoms (a phenyl group, a toluyl group, or a naphthyl group), and an aryl group having 6 to 10 carbon atoms is preferable.

L¹⁹ and L²⁰ are each independently an organic group having a 1,2-naphthoquinonediazido group, and are specifically preferably a 1,2-quinonediazidosulfonyl group such as a 1,2-naphthoquinonediazido-4-sulfonyl group, a 1,2-naphthoquinonediazido-5-sulfonyl group, and a 1,2-naphthoquinonediazido-6-sulfonyl group, and more preferably a 1,2-naphthoquinonediazido-4-sulfonyl group or a 1,2-naphthoquinonediazido-5-sulfonyl group.

p is an integer of 1 to 3, q is an integer of 0 to 4, and 1≤p+q≤5.

J¹⁹ is a single bond, an alkylene group having 1 to 4 carbon atoms, a cycloalkylene group having 3 to 6 carbon atoms, a phenylene group, a group represented by the following formula (c-7-i), a carbonyl group, an ester group, an amide group, or —O—.

Y¹⁹ is a hydrogen atom, an alkyl group having 1 to 6 carbon atoms, or an aryl group having 6 to 10 carbon atoms, and X²⁰ is each independently a group represented by the following formula (c-8-i).

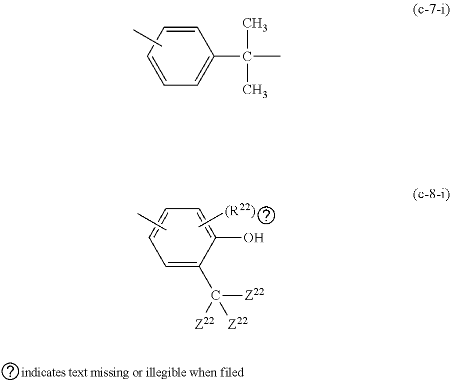

In the above formula (c-8-i), Z²² is each independently an alkyl group having 1 to 6 carbon atoms, a cycloalkyl group having 3 to 6 carbon atoms, or an aryl group having 6 to 10 carbon atoms. R²² is each independently an alkyl group having 1 to 6 carbon atoms, a cycloalkyl group having 3 to 6 carbon atoms, or an alkoxyl group having 1 to 6 carbon atoms, and r is an integer of 0 to 3.

As the acid generating agent used in one aspect of the present invention, other acid generating agents may be used in addition to the compound represented by any of the general formulas (c-1) to (c-8).

Examples of such other acid generating agents include bissulfonyldiazomethanes such as bis(p-toluenesulfonyl)diazomethane, bis(2,4-dimethylphenylsulfonyl)diazomethane, bis(tert-butylsulfonyl)diazomethane, bis(n-butylsulfonyl)diazomethane, bis(isobutylsulfonyl)diazomethane, bis(isopropylsulfonyl)diazomethane, bis(n-propylsulfonyl)diazomethane, bis(cyclohexylsulfonyl)diazomethane, bis(isopropylsulfonyl)diazomethane, 1,3-bis(cyclohexylsulfonylazomethylsulfonyl)propane, 1,4-bis(phenylsulfonylazomethylsulfonyl)butane, 1,6-bis(phenylsulfonylazomethylsulfonyl)hexane, and 1,10-bis(cyclohexylsulfonylazomethylsulfonyl)decane; and halogen-containing triazine derivatives such as 2-(4-methoxyphenyl)-4,6-(bistrichloromethyl)-1,3,5-triazine, 2-(4-methoxynaphthyl)-4,6-(bistrichloromethyl)-1,3,5-triazine, tris(2,3-dibromopropyl)-1,3,5-triazine, and tris(2,3-dibromopropyl) isocyanurate.

### <Other Additives>

The resist auxiliary film composition of one aspect of the present invention may contain other components in addition to the aforementioned components (A) to (C).

Examples of other components include one or more selected from the group consisting of an acid cross-linking agent, an acid diffusion controlling agent, a dissolution accelerator, a dissolution controlling agent, a sensitizing agent, a surfactant, and an organic carboxylic acid or phosphorus oxoacid or a derivative thereof.

Each content of these other components is appropriately selected depending on the type of the component or the resin (A), and is preferably 0.001 to 100 parts by mass, more preferably 0.01 to 70 parts by mass, further preferably 0.1 to 50 parts by mass, and further more preferably 0.3 to 30 parts by mass, per 100 parts by mass of the resin (A) contained in the resist auxiliary film composition.

### (Acid Cross-Linking Agent)

The acid cross-linking agent may be a compound having a cross-linking group capable of cross-linking with the resin (A), and is appropriately selected depending on the type of the resin (A).

Examples of the acid cross-linking agent used in one aspect of the present invention include methylol group-containing compounds such as a methylol group-containing melamine compound, a methylol group-containing benzoguanamine compound, a methylol group-containing urea compound, a methylol group-containing glycoluril compound, and a methylol group-containing phenolic compound; alkoxyalkyl group-containing compounds such as an alkoxyalkyl group-containing melamine compound, an alkoxyalkyl group-containing benzoguanamine compound, an alkoxyalkyl group-containing urea compound, an alkoxyalkyl group-containing glycoluril compound, and an alkoxyalkyl group-containing phenolic compound; carboxymethyl group-containing compounds such as a carboxymethyl group-containing melamine compound, a carboxymethyl group-containing benzoguanamine compound, a carboxymethyl group-containing urea compound, a carboxymethyl group-containing glycoluril compound, and a carboxymethyl group-containing phenolic compound; and epoxy compounds such as a bisphenol An epoxy compound, a bisphenol F epoxy compound, a bisphenol S epoxy compound, a novolac resin epoxy compound, a resol resin epoxy compound, and a poly(hydroxystyrene) epoxy compound.

These acid cross-linking agents may be used singly or in combination of two or more thereof.

### (Acid Diffusion Controlling Agent)

The acid diffusion controlling agent is an additive that functions to, for example, control the diffusion of an acid generated from the acid generating agent in the resist auxiliary film to inhibit an unpleasant chemical reaction.

Examples of the acid diffusion controlling agent used in one aspect of the present invention include, but are not particularly limited to, radiation decomposable basic compounds such as a nitrogen atom-containing basic compound, a basic sulfonium compound, and a basic iodonium compound.

These acid diffusion controlling agents may be used singly or in combination of two or more thereof.

### (Dissolution Accelerator)

The dissolution accelerator is an additive that functions to increase the solubility of the resin (A) in a developer to moderately increase the dissolution rate of the resin (A) in development.

Examples of the dissolution accelerator used in one aspect of the present invention include, but are not particularly limited to, phenolic compounds such as bisphenols and tris(hydroxyphenyl) methane.

These dissolution accelerators may be used singly or in combination of two or more thereof.

### (Dissolution Controlling Agent)

The dissolution controlling agent is an additive that functions to control the solubility of the resin (A) to moderately reduce the dissolution rate in development if the solubility thereof in the developer is too high.

Examples of the dissolution controlling agent used in one aspect of the present invention include, but are not particularly limited to, aromatic hydrocarbons such as phenanthrene, anthracene, and acenaphthene; ketones such as acetophenone, benzophenone, and phenylnaphthylketone; and sulfones such as methyl phenyl sulfone, diphenyl sulfone, and dinaphthyl sulfone.

These dissolution controlling agents may be used singly or in combination of two or more thereof.

### (Sensitizing Agent)

The sensitizing agent is an additive that functions to absorb the energy of radiation irradiated and transmit the energy to the acid generating agent to thereby increase the amount of acid generated. The sensitizing agent is also an additive that functions to absorb light with a specific wavelength.

Examples of the sensitizing agent used in one aspect of the present invention include benzophenones, biacetyls, pyrenes, phenothiazines, and fluorenes.

These sensitizing agents may be used singly or in combination of two or more thereof.

### (Surfactant)

The surfactant is an additive that functions to improve, for example, the applicability, striation, and developability of the resist auxiliary film composition.

The surfactant used in one aspect of the present invention may be any of an anionic surfactant, a cationic surfactant, a nonionic surfactant, and an amphoteric surfactant, and a nonionic surfactant is preferable. Examples of the nonionic surfactant include polyoxyethylene higher alkyl ethers, polyoxyethylene higher alkylphenyl ethers, and higher fatty acid diesters of polyethylene glycol.

These surfactants may be used singly or in combination of two or more thereof.

### (Organic Carboxylic Acid or Phosphorus Oxoacid or Derivative Thereof)

The organic carboxylic acid or phosphorus oxoacid or a derivative thereof is an additive that functions to, for example, prevent deterioration in sensibility and improve the resist pattern shape and stability in post exposure delay.

Examples of the organic carboxylic acid used in one aspect of the present invention include, but are not particularly limited to, malonic acid, citric acid, malic acid, succinic acid, benzoic acid, and salicylic acid. Examples of the phosphorus oxoacid or a derivative thereof include phosphoric acids or derivatives such as esters thereof, such as phosphoric acid, phosphoric acid di-n-butyl ester, and phosphoric acid diphenyl ester: phosphonic acid or derivatives such as esters thereof, such as phosphonic acid, phosphonic acid dimethyl ester, phosphonic acid di-n-butyl ester, phenylphosphonic acid, phosphonic acid diphenyl ester, and phosphonic acid dibenzyl ester; and phosphinic acid and derivatives such as esters thereof, such as phosphinic acid and phenylphosphinic acid.

These may be used singly or in combination of two or more.

### (Other Components)

The resist auxiliary film composition of one aspect of the present invention may contain a dye, a pigment, an adhesion aid, a halation preventing agent, a storage stabilizing agent, a defoaming agent, a shape improver, or the others, in addition to the aforementioned other components.

### [Method for Forming Pattern]

Further, one of the present embodiment is a method for forming a pattern, and the pattern formation method includes step (A-1) of forming a resist underlayer film on a substrate by using the resist auxiliary film composition of the present embodiment, step (A-2) of forming at least one photoresist layer on the resist underlayer film, and step (A-3) of irradiating a predetermined region of the photoresist layer with radiation after step (A-2), followed by developing.

As mentioned above, the resist auxiliary film composition of one aspect of the present invention can form a thick resist auxiliary film (here, a resist underlayer film) suitable for the manufacture of various devices, although the content of the active component including the resin is limited to a predetermined value or less.

When the resist auxiliary film composition is used as a photoresist underlayer film material for a spin-on-carbon film used in a two-layer resist method or a three-layer resist method, the pattern formation method includes step (B-1) of forming a resist underlayer film by using the resist auxiliary film composition of the present embodiment, step (B-2) of forming a resist intermediate layer film on the resist underlayer film, step (B-3) of forming at least one photoresist layer on the resist intermediate layer film, step (B-4) of irradiating a predetermined region of the photoresist layer with radiation after step (B-3), followed by developing to form a resist pattern, and step (B-5) of etching the resist intermediate layer film by using the resist pattern as a mask after step (B-4), etching the resist underlayer film by using the obtained resist intermediate layer film pattern as an etching mask, and etching the substrate by using the obtained resist underlayer film pattern as an etching mask to form a pattern on the substrate.

The formation method of the aforementioned resist underlayer film is not particularly limited, as long as the resist underlayer film is formed from the resist auxiliary film composition of the present embodiment, and a known method can be applied. For example, the resist underlayer film can be formed by applying the resist auxiliary film composition of the present embodiment by a known coating method or printing method, such as spin coating and screen printing, on a substrate, and then removing the organic solvent by volatilization or the like.

Upon forming the resist underlayer film, baking is preferably performed to suppress the occurrence of phenomenon of mixing with the upper layer resist and also to promote the cross-linking reaction. In this case, the baking temperature is not particularly limited, and is preferably within the range of 80 to 600° C., and more preferably 200 to 400° C. The baking time is also not particularly limited, and is preferably within the range of 10 to 300 seconds. The thickness of the resist underlayer film can be appropriately selected depending on the required performance, and is usually preferably, but not particularly limited to, 3 to 20,000 nm, more preferably 10 to 15,000 nm, and further preferably 50 to 1000 nm.

When the resist auxiliary film composition is used as an antireflection film for KrF excimer laser or ArF excimer laser or a photoresist underlayer film material for EUV lithography after the resist underlayer film is formed on a substrate, a single-layer resist layer is preferably produced thereon. In this case, a known material can be used as the photoresist material for forming the resist layer.

When the resist auxiliary film composition is used as a photoresist underlayer film material for a spin-on-carbon film used in a two-layer resist method or a three-layer resist method after the resist underlayer film is formed on a substrate, a silicon-containing resist layer or a usual single-layer resist composed of hydrocarbon is preferably produced on the resist underlayer film in the case of a two-layer process, and, in the case of a three-layer process, a silicon-containing intermediate layer is preferably produced on the resist underlayer film, followed by producing a further single-layer resist layer containing no silicon on the silicon-containing intermediate laver. In these cases, known materials can be used as the photoresist material for forming the resist layer.

For the silicon-containing resist material for the two-layer process, a positive type photoresist material is preferably used that contains a silicon atom-containing polymer, such as a polysilsesquioxane derivative or a vinyl silane derivative, used as a base polymer, and furthermore, an organic solvent, an acid generating agent, and if necessary, a basic compound, in view of oxygen gas etching resistance. Here, a known polymer that is used in this kind of resist material can be used as the silicon atom-containing polymer.

For the silicon-containing intermediate layer for the three-layer process, a polysilsesquioxane-based intermediate layer is preferably used. By the intermediate layer serving as the antireflection film, reflection tends to be effectively suppressed. For example, when a material containing a large amount of aromatic groups and having high substrate etching resistance is used as the resist underlayer film in a process of exposure at 193 nm, the k value tends to be high and substrate reflection tends to be high. However, by suppressing the reflection by the intermediate layer, the substrate reflection can be reduced to 0.5% or less. For the intermediate layer having such an antireflection effect, polysilsesquioxane in which a phenyl group, or a light absorbing group having a silicon-silicon bond is introduced and which is cross-linked by an acid or heat is preferably used for exposure at 193 nm, but not limited thereto.

An intermediate layer formed by a chemical vapour deposition (CVD) method can also be used. As the intermediate layer produced by the CVD method and having a large effect as the antireflection film, a SiON film is known, for example, but not limited thereto. Typically, the formation of an intermediate layer by a wet process such as spin coating and screen printing is more convenient and advantageous in terms of cost than the formation by the CVD method. The upper layer resist in the three-layer process may be either positive type or negative type, and those usually used as the single-layer resist can be used.

When the resist layer is formed from the above photoresist material, a wet process such as spin coating and screen printing is preferably used as in the case of forming the above resist underlayer film. After the resist material is applied by spin coating or the like, prebaking is usually performed. The prebaking is preferably performed at 80 to 180° C. in the range of 10 to 300 seconds. Thereafter, exposure, post exposure baking (PEB), and development are performed according to a conventional method, thereby obtaining a resist pattern. The thickness of the resist film is not particularly limited, and is typically, preferably 10 to 50,000 nm, more preferably 20 to 20,000 nm, and further preferably 50 to 15,000 nm.

The exposure light may be appropriately selected depending on the photoresist material to be used. Typically, examples thereof include high energy rays having a wavelength of 300 nm or less, specifically, an excimer laser of 248 nm, 193 nm, or 157 nm, a soft x-ray of 3 to 20 nm, an electron beam, and an X-ray.

In the resist pattern formed by the aforementioned method, pattern collapse is suppressed by the resist underlayer film according to the present embodiment. Thus, use of the resist underlayer film according to the present embodiment enables a finer pattern to be obtained, and may reduce the amount of exposure required to obtain the resist pattern.

Then, etching is performed by using the obtained resist pattern as a mask. Gas etching is preferably used for the etching of the resist underlayer film in the two-layer process. Etching using oxygen gas is suitable as gas etching. In addition to oxygen gas, an inert gas such as He or Ar, or CO, CO₂, NH₃, SO₂, N₂, NO₂, or H₂ gas may be added. Alternatively, gas etching can also be performed by using only CO, CO₂, NH₃, N₂, NO₂, or H₂ gas, without using oxygen gas. In particular, the latter gases are preferably used for side wall protection to prevent the undercut of pattern side walls.

On the other hand, gas etching is preferably used also in the etching of the intermediate layer in the three-layer process. The same gas etching as described for the aforementioned two-layer process is applicable to the gas etching. Especially, the intermediate layer in the three-layer process is preferably processed with chlorofluorocarbon-based gas and a resist pattern as a mask. Thereafter, the resist underlayer film can be processed by, for example, oxygen gas etching using the intermediate layer pattern as a mask, as mentioned above.

Here, when an inorganic hard mask intermediate layer film is formed as the intermediate layer, a silicon oxide film, a silicon nitride film, or a silicon oxynitride film (SiON film) is formed by the CVD method, the ALD method, or the like. As the method for forming a nitride film, the method described in, for example, Japanese Patent Laid-Open No. 2002-334869 or WO 2004/066377 can be used, but not limited thereto. Although a photoresist film can be directly formed on such a resist intermediate layer film, an organic antireflection film (BARC) may be formed on the resist intermediate layer film by spin coating, and a photoresist film may be formed thereon.

A polysilsesquioxane-based intermediate layer is also preferably used as the intermediate layer. By the resist intermediate layer film serving as the antireflection film, reflection tends to be effectively suppressed. Examples of the specific material of the polysilsesquioxane-based intermediate layer that can be used include, but not limited to, those described in Japanese Patent Laid-Open No. 2007-226170 and Japanese Patent Laid-Open No. 2007-226204.

The subsequent etching of the substrate can also be performed by a conventional method. For example, when the substrate is SiO₂ or SiN, etching mainly using chlorofluorocarbon-based gas can be performed, and when the substrate is p-Si, Al, or W, etching mainly using chlorine-based or bromine-based gas can be performed. When the substrate is etched with chlorofluorocarbon-based gas, the silicon-containing resist of the two-layer resist process and the silicon-containing intermediate layer of the three-layer process are stripped simultaneously with substrate processing. On the other hand, when the substrate is etched with chlorine-based or bromine-based gas, the silicon-containing resist layer or the silicon-containing intermediate layer is separately stripped, and is typically stripped by dry etching with chlorofluorocarbon-based gas after substrate processing.

The resist underlayer film according to the present embodiment is characterized in that the film is excellent in the etching resistance of these substrates. A known substrate can be appropriately selected and used, and examples thereof include, but are not particularly limited to, Si, α-Si, p-Si, SiO₂, SiN, SiON, W, TiN, and Al. The substrate may be a laminate having a film to be processed (substrate to be processed) on the base material (support). Examples of such a film to be processed include various Low-k films of, for example, Si, SiO₂, SiON, SiN, p-Si, α-Si, W, W—Si, Al, Cu, and Al—Si, and stopper films thereof, and a material different from that of the base material (support) is usually used. The thickness of the substrate to be processed or the film to be processed is not particularly limited, and is usually, preferably about 50 to 1,000,000 nm, and more preferably 75 to 500,000 nm.

The resist auxiliary film composition of one aspect of the present invention can also be used for the formation of the resist intermediate layer film. The method for forming a pattern of another embodiment of the present invention includes step (B-1) of forming a resist underlayer film on a substrate, step (B-2) of forming a resist intermediate layer film by using the resist auxiliary film composition of the embodiment of the present invention on the resist underlayer film, step (B-3) of forming at least one photoresist layer on the resist intermediate layer film, step (B-4) of irradiating a predetermined region of the photoresist layer with radiation after step (B-3), followed by developing to form a resist pattern, and step (B-5) of etching the resist intermediate layer film by using the resist pattern as a mask after step (B-4), etching the resist underlayer film by using the obtained resist intermediate layer film pattern as an etching mask, and etching the substrate by using the obtained resist underlayer film pattern as an etching mask to form a pattern on the substrate.

### EXAMPLES

Hereinafter, the present invention will be described by way of Examples, but the present invention is not limited by these Examples in any way. The measurement values in Examples were found by using the following method or apparatus.

### (1) Film Thickness of Coating Film

The film thickness of a coating film formed from the resist auxiliary film composition was measured using a film thicknesses measurement system (apparatus name “F20”, manufactured by Filmetrics, Inc.) in a constant-temperature constant-humidity chamber with a temperature of 23° C. and a humidity of 50% (relative humidity).

### (2) Content of Constitutional Unit of Resin

The content of the constitutional unit of a resin was measured by performing 1024 scans in the quantitative mode of ¹³C using ¹³C-NMR (model name “JNM-ECA500”, manufactured by JEOL Ltd., 125 MHZ) with chloroform-d as a solvent.

### (3) Weight Average Molecular Weight (Mw), Number Average Molecular Weight (Mn), and Molecular Weight Distribution (Mw/Mn) of Resin

Mw and Mn of the resin were measured, in terms of polystyrene as a standard, by gel permeation chromatography (GPC) under the following conditions.
Apparatus name: LaChrom series manufactured by Hitachi, Ltd.
Detector: RI detector L-2490
Column: two TSKgel GMHHR-M columns+guard column HHR-H manufactured by Tosoh Corporation
Solvent: THF (with a stabilizer)
Flow rate: 1 mL/min
Column temperature: 40° C.

Then, the ratio of the calculated Mw to Mn [Mw/Mn] of the resin was calculated as the value of the molecular weight distribution of the resin.

The solvents used in the following Examples and Comparative Examples were as follows.

### <Component (B1)>

HBM: methyl 2-hydroxyisobutyrate, a compound in which R¹ is a methyl group in the general formula (b-1).
iPHIB: isopropyl 2-hydroxyisobutyrate, a compound in which R¹ is an i-propyl group in the general formula (b-1).
iBHIB: isobutyl 2-hydroxyisobutyrate, a compound in which R¹ is an i-butyl group in the general formula (b-1).
nBHIB: n-butyl 2-hydroxyisobutyrate, a compound in which R¹ is an n-butyl group in the general formula (b-1).

### <Component (B2)>

PGMEA: propylene glycol monomethyl ether acetate
MMP: methyl 3-methoxypropionate
nBuOAc: n-butyl acetate
EL: ethyl lactate

### [Resist Auxiliary Film Composition Containing Novolac Resin]

### Examples 1a to 47a, Comparative Examples 1a to 6a

A cresol novolac resin obtained by mixing “EP4080G” and “EP4050G” (both manufactured by ASAHI YUKIZAI CORPORATION) in the ratio of 1:1 (mass ratio) was used as a liquid crystal resin.

84 Parts by mass of the above cresol novolac resin and 16 parts by mass of a diazonaphthoquinone photosensitizer (trade name “DTEP-350” manufactured by DAITO CHEMIX Co., Ltd.) were mixed into a solvent of the kind and the blending ratio described in Table 1 and dissolved therein to prepare a resist auxiliary film composition having an active component (the above cresol novolac resin and photosensitizer) concentration described in Table 1 and Table 2.

Then, a coating film was formed from the prepared resist auxiliary film composition on a silicon wafer by spin coating at 1600 rpm, and the coating film was subjected to prebaking at 110° C. for 90 seconds to form a resist auxiliary film. The film thicknesses were measured at randomly selected 5 points on the resist auxiliary film, and the average value of the film thicknesses at 5 points was calculated as the average film thickness. The results are shown in Table 1 and Table 2.

TABLE 1

| | Kind and amount | | | |
|---|---|---|---|---|
| | of solvent blended | | | |
| | | Amount | Active | |
| | | blended | component | Film |
| | | (% by | concentration | thickness |
| | Kind | mass) | (% by mass) | (nm) |
| Example 1a | HBM | 100 | 25 | 2920 |
| Example 2a | | | 20 | 1680 |
| Example 3a | | | 18 | 1350 |
| Example 4a | | | 16 | 1050 |
| Example 5a | iPHIB | 100 | 22 | 2100 |
| Example 6a | | | 20 | 1550 |
| Example 7a | | | 18 | 1200 |
| Example 8a | iBHIB | 100 | 25 | 2850 |
| Example 9a | | | 22 | 1850 |
| Example 10a | | | 20 | 1450 |
| Example 11a | | | 18 | 1030 |
| Example 12a | nBHIB | 100 | 25 | 2450 |
| Example 13a | | | 22 | 1550 |
| Example 14a | | | 20 | 1200 |
| Comparative | PGMEA | 100 | 25 | 1850 |
| Example 1a | | | | |
| Comparative | | | 23 | 1480 |
| Example 2a | | | | |
| Comparative | | | 21 | 1180 |
| Example 3a | | | | |
| Comparative | EL | 100 | 25 | 2050 |
| Example 4a | | | | |
| Comparative | | | 23 | 1650 |
| Example 5a | | | | |
| Comparative | | | 21 | 1300 |
| Example 6a | | | | |

TABLE 2

| | Kind and amount of solvent blended | | | | Active | Average |
|---|---|---|---|---|---|---|
| | | Amount | | Amount | component | film |
| | | blended | | blended | concentration | thickness |
| | Kind | (% by mass) | Kind | (% by mass) | (% by mass) | (nm) |
| Example 15a | HBM | 70 | PGMEA | 30 | 25 | 2560 |
| Example 16a | | | | | 22 | 1850 |
| Example 17a | | | | | 20 | 1460 |
| Example 18a | HBM | 30 | PGMEA | 70 | 25 | 2090 |
| Example 19a | | | | | 22 | 1550 |
| Example 20a | | | | | 20 | 1250 |
| Example 21a | HBM | 70 | MMP | 30 | 25 | 2390 |
| Example 22a | | | | | 22 | 1680 |
| Example 23a | | | | | 20 | 1390 |
| Example 24a | HBM | 30 | MMP | 70 | 25 | 1870 |
| Example 25a | | | | | 22 | 1360 |
| Example 26a | | | | | 20 | 1110 |
| Example 27a | HBM | 80 | nBuOAc | 20 | 25 | 2890 |
| Example 28a | | | | | 22 | 2040 |
| Example 29a | | | | | 20 | 1650 |
| Example 30a | iPHIB | 70 | PGMEA | 30 | 25 | 2450 |
| Example 31a | | | | | 22 | 1710 |
| Example 32a | | | | | 20 | 1350 |
| Example 33a | iPHIB | 30 | PGMEA | 70 | 25 | 2080 |
| Example 34a | | | | | 22 | 1490 |
| Example 35a | | | | | 20 | 1190 |
| Example 36a | iBHIB | 70 | PGMEA | 30 | 25 | 2670 |
| Example 37a | | | | | 22 | 1780 |
| Example 38a | | | | | 20 | 1280 |
| Example 39a | iBHIB | 30 | PGMEA | 70 | 25 | 2060 |
| Example 40a | | | | | 22 | 1460 |
| Example 41a | | | | | 20 | 1160 |
| Example 42a | nBHIB | 70 | PGMEA | 30 | 25 | 2560 |
| Example 43a | | | | | 22 | 1600 |
| Example 44a | | | | | 20 | 1250 |
| Example 45a | nBHIB | 30 | PGMEA | 70 | 25 | 2010 |
| Example 46a | | | | | 22 | 1360 |
| Example 47a | | | | | 20 | 1130 |

It is found from Table 1 that the resist auxiliary film compositions prepared in Examples 1a to 14a can form thick resist auxiliary films as compared with the resist auxiliary film compositions of Comparative Examples 1b to 6b each having a comparable resin concentration.

It is also found from Table 2 that the resist auxiliary film compositions prepared in Examples 15a to 47a can form thick resist auxiliary films, although the content of the novolac resin is as low as 20 to 25% by mass.

### [Resist Auxiliary Film Composition Containing Ethylenically Unsaturated Resin (0)]

### Examples 1b to 35b, Comparative Examples 1b to 19b

A copolymer having a constitutional unit of hydroxystyrene/t-butyl acrylate=2/1 (molar ratio) (manufactured by Maruzen Petrochemical Co., Ltd., Mw=20,000) was used as the ethylenically unsaturated resin (0).

The above copolymer and a mixed solvent of the kind and the blending ratio shown in Table 3 and Table 4 were mixed to prepare a resist auxiliary film composition having an active component (ethylenically unsaturated resin (0)) concentration described in Table 3 and Table 4.

Then, a coating film was formed from the prepared resist auxiliary film composition on a silicon wafer by spin coating at 1600 rpm, and the coating film was subjected to prebaking at 110° C. for 90 seconds to form a resist auxiliary film. The film thicknesses were measured at randomly selected 5 points on the resist auxiliary film, and the average value of the film thicknesses at 5 points was calculated as the average film thickness. The results are shown in Table 3 and Table 4.

TABLE 3

| | | Active | |
|---|---|---|---|
| | | component | Average film |
| | | concentration | thickness |
| | Kind of solvent | (% by mass) | (nm) |
| Example 1b | HBM | 35 | 25700 |
| Example 2b | | 30 | 10600 |
| Example 3b | | 20 | 2430 |
| Example 4b | | 15 | 1030 |
| Example 5b | | 10 | 547 |
| Example 6b | iPHIB | 35 | 26000 |
| Example 7b | | 30 | 11000 |
| Example 8b | | 20 | 2110 |
| Example 9b | | 15 | 834 |
| Example 10b | | 10 | 436 |
| Example 11b | iBHIB | 35 | 35700 |
| Example 12b | | 30 | 15200 |
| Example 13b | | 20 | 2320 |
| Example 14b | | 15 | 811 |
| Example 15b | | 10 | 418 |
| Example 16b | nBHIB | 35 | 26900 |
| Example 17b | | 30 | 12300 |
| Example 18b | | 20 | 2100 |
| Example 19b | | 15 | 786 |
| Example 20b | | 10 | 400 |
| Example 27b | Mixed solvent of | 35 | 18200 |
| Example 28b | HBM/nBuOAc = 8/2 | 30 | 8470 |
| Example 29b | (mass ratio) | 20 | 2350 |
| Example 30b | | 10 | 716 |
| Example 31b | Mixed solvent of | 40 | 25000 |
| Example 32b | HBM/MMP = 5/5 | 35 | 13500 |
| Example 33b | (mass ratio) | 30 | 5680 |
| Example 34b | | 20 | 2170 |
| Example 35b | | 10 | 562 |

TABLE 4

| | | Active | |
|---|---|---|---|
| | | component | Average film |
| | Kind of | concentration | thickness |
| | solvent | (% by mass) | (nm) |
| Comparative | PGMEA | 40 | 21700 |
| Example 1b | | | |
| Comparative | | 35 | 11200 |
| Example 2b | | | |
| Comparative | | 30 | 5050 |
| Example 3b | | | |
| Comparative | | 20 | 1430 |
| Example 4b | | | |
| Comparative | | 15 | 555 |
| Example 5b | | | |
| Comparative | | 10 | 444 |
| Example 6b | | | |
| Comparative | MMP | 30 | 4190 |
| Example 7b | | | |
| Comparative | | 20 | 1350 |
| Example 8b | | | |
| Comparative | | 15 | 676 |
| Example 9b | | | |
| Comparative | | 10 | 378 |
| Example 10b | | | |
| Comparative | nBuOAc | 30 | 7380 |
| Example 11b | | | |
| Comparative | | 20 | 2070 |
| Example 12b | | | |
| Comparative | | 15 | 1010 |
| Example 13b | | | |
| Comparative | | 10 | 481 |
| Example 14b | | | |
| Comparative | EL | 35 | 15300 |
| Example 15b | | | |
| Comparative | | 30 | 7700 |
| Example 16b | | | |
| Comparative | | 20 | 1870 |
| Example 17b | | | |
| Comparative | | 15 | 777 |
| Example 18b | | | |
| Comparative | | 10 | 429 |
| Example 19b | | | |

It is found from Table 3 and Table 4 that the resist auxiliary film compositions prepared in Examples 1b to 35b can form thick resist auxiliary films as compared with the resist auxiliary film compositions of Comparative Examples 1b to 19b having the same resin concentration.

[Resist Auxiliary Film Composition Containing Ethylenically Unsaturated Resins (i) to (vi)]

### Synthetic Examples 1 to 6 (Synthesis of Ethylenically Unsaturated Resins (i) to (vi))

### (1) Monomer as Starting Material

In the synthesis of the ethylenically unsaturated resins (i) to (vi), the following monomers as starting materials were used. The structure of each monomer as the starting material is as shown in Table 5.
EADM: 2-ethyl-2-adamantyl methacrylate
MADM: 2-methyl-2-adamantyl methacrylate
NML: 2-methacryloxy-4-oxatricyclo[4.2.1.0^{3.7}]nonan-5-one
GBLM: α-methacryloxy-γ-butyrolactone
HADM: 3-hydroxy-1-adamantyl methacrylate

TABLE 5

| Monomer | | | |
|---|---|---|---|
| as starting | | | |
| material | EADM | MADM | NML |
| Compound | 2-Ethyl-2-adamantyl | 2-Methyl-2-adamantyl | 2-Methacloyloxy-4- |
| name | methacrylate | methacrylate | oxatricyclo- |
| | | | [4.2.10^{3.7}]nonan-5-one |
| Structure | 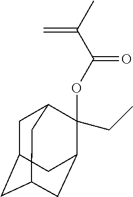 [SMILES: C=C(C)C(=O)OC1(CC)C2CC3CC(C2)CC1C3] | 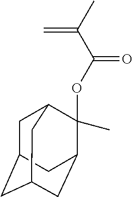 [SMILES: C=C(C)C(=O)OC1(C)C2CC3CC(C2)CC1C3] | 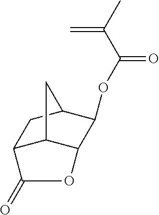 [SMILES: C=C(C)C(=O)OC1C2CC3C(=O)OC1C3C2] |

| Monomer | | |
|---|---|---|
| as starting | | |
| material | GBLM | HADM |
| Compound | α-Methacloyloxy-γ- | 3-Hydroxy-1-adamantyl |
| name | butyrolactone | methacrylate |
| Structure | 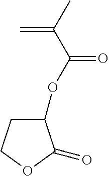 | 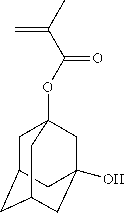 [SMILES: C=C(C)C(=O)OC12CC3CC(CC(O)(C3)C1)C2] |

### (2) Synthesis of Ethylenically Unsaturated Resins (i) to (vi)

In a 300 mL round bottomed flask, 10 g in total of the monomers as starting materials were blended according to the kind and the blending ratio described in Table 6, and 300 g of tetrahydrofuran (manufactured by Wako Pure Chemical Industries, Ltd., guaranteed reagent, free from stabilizer) was further added, followed by stirring. Then, the mixture was degassed for 30 minutes in a nitrogen stream. After degassing, 0.95 g of 2,2′-azobis(isobutyronitrile) (manufactured by Tokyo Kasei Kogyo Co., Ltd., reagent) was added, and a polymerization reaction was conducted at 60° C. in a nitrogen stream so as to obtain a resin having a desired molecular weight.

After the completion of the reaction, the reaction solution cooled to room temperature (25° C.) was added dropwise to a large excess amount of hexane to precipitate a polymerization product. The precipitated polymerization product was collected by filtering, and the obtained solid was washed with methanol and then dried under reduced pressure at 50° C. for 24 hours to obtain each of the intended ethylenically unsaturated resins (i) to (vi).

The content of each constitutional unit, and the Mw, Mn, and Mw/Mn of the obtained ethylenically unsaturated resins (i) to (vi) were measured and calculated based on the aforementioned measurement method. The results are shown in Table 6.

TABLE 6

| | Formulation of monomers as | | | | | Content of constitutional | | | | | | Molecular | | |
|---|---|---|---|---|---|---|---|---|---|---|---|---|---|---|
| Acrylic | starting materials (molar ratio) | | | | | unit of resin (mol %) | | | | | | weight of resin | | |
| resin | EADM | MADM | NML | GBLM | HADM | EADM | MADM | NML | GBLM | HADM | Total | Mw | Mn | Mw/Mn |
| Resin (i) | 40 | | 40 | | 20 | 32.8 | | 38.3 | | 28.9 | 100 | 5940 | 3260 | 1.82 |
| Resin (ii) | 40 | | | 40 | 20 | 30.7 | | | 43.9 | 25.4 | 100 | 9850 | 4820 | 2.04 |
| Resin (iii) | | 40 | 40 | | 20 | | 38.0 | 37.7 | | 24.3 | 100 | 6480 | 3160 | 2.05 |
| Resin (iv) | | 40 | | 40 | 20 | | 23.7 | | 44.9 | 31.4 | 100 | 15800 | 6820 | 2.32 |
| Resin (v) | | 40 | 40 | | 20 | | 41.6 | 33.0 | | 25.4 | 100 | 14200 | 7260 | 1.96 |
| Resin (vi) | | 40 | | 60 | | | 25.4 | | 74.6 | | 100 | 11500 | 3770 | 3.05 |

### Examples 1c to 18c, Comparative Examples 1c to 12c

Any of the ethylenically unsaturated resins (i) to (vi) obtained in the above Synthetic Examples 1 to 6 was mixed with a solvent of the kind shown in Tables 7 and 8 to prepare a resist auxiliary film composition having an active component (ethylenically unsaturated resins (i) to (vi)) concentration described in Tables 7 and 8.

Then, a coating film was formed from the prepared resist auxiliary film composition on a silicon wafer by spin coating at 3000 rpm, and the coating film was subjected to prebaking at 90° C. for 60 seconds to form a resist auxiliary film. The film thicknesses were measured at randomly selected 5 points on the resist auxiliary film, and the average value of the film thicknesses at 5 points was calculated as the average film thickness. The results are shown in Table 7 and Table 8.

TABLE 7

| | | | Active | |
|---|---|---|---|---|
| | | | component | Average film |
| | Acrylic | Kind of | concentration | thickness |
| | resin | solvent | (% by mass) | (nm) |
| Example 1c | Resin (i) | HBM | 10 | 343 |
| Example 2c | | | 5 | 131 |
| Example 3c | | | 2 | 45.7 |
| Example 4c | Resin (ii) | HBM | 10 | 373 |
| Example 5c | | | 5 | 132 |
| Example 6c | | | 2 | 42.1 |
| Example 7c | Resin (iii) | HBM | 10 | 361 |
| Example 8c | | | 5 | 142 |
| Example 9c | | | 2 | 44.9 |
| Example 10c | Resin (iv) | HBM | 10 | 401 |
| Example 11c | | | 5 | 142 |
| Example 12c | | | 2 | 44.7 |
| Example 13c | Resin (v) | HBM | 10 | 420 |
| Example 14c | | | 5 | 143 |
| Example 15c | | | 2 | 44.1 |
| Example 16c | Resin (vi) | HBM | 10 | 343 |
| Example 17c | | | 5 | 133 |
| Example 18c | | | 2 | 45.4 |

TABLE 8

| | | | Active | |
|---|---|---|---|---|
| | | | component | Average film |
| | Acrylic | Kind of | concentration | thickness |
| | resin | solvent | (% by mass) | (nm) |
| Comparative | Resin (i) | PGMEA | 10 | 215 |
| Example 1c | | | | |
| Comparative | | | 5 | 95.7 |
| Example 2c | | | | |
| Comparative | | | 2 | 28.9 |
| Example 3c | | | | |
| Comparative | Resin (ii) | PGMEA | 10 | 237 |
| Example 4c | | | | |
| Comparative | | | 5 | 90.0 |
| Example 5c | | | | |
| Comparative | | | 2 | 28.1 |
| Example 6c | | | | |
| Comparative | Resin (iii) | PGMEA | 10 | 232 |
| Example 7c | | | | |
| Comparative | | | 5 | 92.3 |
| Example 8c | | | | |
| Comparative | | | 2 | 33.9 |
| Example 9c | | | | |
| Comparative | Resin (iv) | PGMEA | 10 | 256 |
| Example 10c | | | | |
| Comparative | | | 5 | 97.2 |
| Example 11c | | | | |
| Comparative | | | 2 | 29.8 |
| Example 12c | | | | |

It is found from Table 7 and Table 8 that the resist auxiliary film compositions prepared in Examples 1c to 18c can form thick resist auxiliary films as compared with the resist auxiliary film compositions of Comparative Examples 1c to 12c having the same resin concentration.

### Example 1d, Comparative Example 1d

### (Preparation of Underlayer Film Composition)

An underlayer film composition was prepared so as to have the composition shown in Table 9. The following were used as the polymer, the acid generating agent, the cross-linking agent, and the organic solvent.

Polymer: The resin of the formula (R1-1) was prepared as follows. Specifically, 30 g of 4,4-biphenol, 15 g of 4-biphenylaldehyde, and 100 mL of butyl acetate were blended, and 3.9 g of p-toluenesulfonic acid was added thereto to prepare a reaction solution. The reaction solution was reacted by stirring at 90° C. for 3 hours. Then, the reaction solution was concentrated, and the reaction solution was added dropwise into 400 mL of n-heptane. In this manner, the obtained resin product was solidified and purified, and the produced white powder was collected by filtering and dried at 40° C. under reduced pressure overnight to obtain a resin represented by the following formula (R1-1).

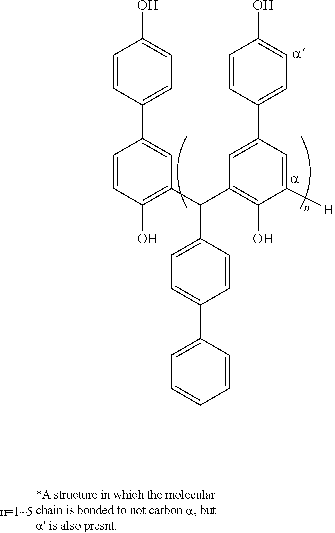

Acid generating agent: di-tertiary butyl diphenyliodonium nonafluoromethanesulfonate (DTDPI) manufactured by Midori Kagaku Co., Ltd.
Cross-linking agent: NIKALACMX270 (NIKALAC) manufactured by Sanwa Chemical Co., Ltd.
TMOM-BP (a compound represented by the following formula) manufactured by Honshu Chemical Industry Co., Ltd.

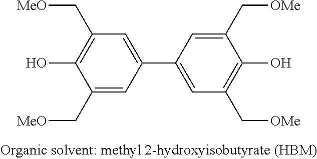 [SMILES: COCc1cc(-c2cc(COC)c(O)c(COC)c2)cc(COC)c1O]

TABLE 9

| | Polymer | Solvent | Acid | Cross- |
|---|---|---|---|---|
| | (parts | (parts | generating agent | linking agent |
| | by mass) | by mass) | (parts by mass) | (parts by mass) |
| Example 1d | R1-1 | HBM | DTDPI | TMOM-BP |
| | (10) | (90) | (0.5) | (0.5) |

Then, the underlayer film composition prepared in Example 1d was applied to a SiO₂ substrate having a film thickness of 300 nm, baked at 240° C. for 60 seconds, and further baked at 400° C. for 120 seconds to form an underlayer film having a film thickness of 85 nm. A resist solution for ArF was applied to the underlayer film, and baked at 130° C. for 60 seconds to form a photoresist layer having a film thickness of 140 nm.

As the resist solution for ArF, a solution prepared by blending 5 parts by mass of the resin of the following formula (1d), 1 part by mass of triphenylsulfonium nonafluoromethanesulfonate, 2 parts by mass of tributylamine, and 92 parts by mass of PGMEA was used.

The resin of the following formula (1d) was prepared as follows. Specifically, 4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile were dissolved in 80 mL of tetrahydrofuran to obtain a reaction solution. The reaction solution was polymerized for 22 hours by holding the reaction temperature at 63° C. under nitrogen atmosphere, and then, the reaction solution was added dropwise into 400 mL of n-hexane. In this manner, the obtained resin product was solidified and purified, and the produced white powder was collected by filtering and dried at 40° C. under reduced pressure overnight to obtain a resin represented by the following formula (1d).

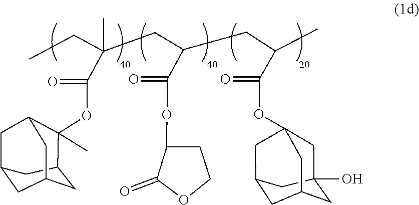

(In the formula (1d), 40, 40, and 20 indicate the ratio between the constitutional units, and do not indicate the block copolymer.)

Then, the above photoresist layer was exposed using an electron beam lithography apparatus (manufactured by ELIONIX INC.; ELS-7500, 50 keV), baked (PEB) at 115° C. for 90 seconds, and developed with a 2.38% by mass aqueous tetramethylammonium hydroxide (TMAH) solution for 60 seconds to obtain a positive type resist pattern.

### Comparative Example 1d

A positive type resist pattern was obtained in the same manner as in Example 1d, except that no resist underlayer film was formed, or in other words, a photoresist layer was directly formed on a SiO₂ substrate.

### [Evaluation]

For each of Example 1d and Comparative Example 1d, the shapes of the obtained resist patterns with 40 nmL/S (1:1) and 80 nmL/S (1:1) were observed using an electron microscope “S-4800” manufactured by Hitachi, Ltd. Regarding the shape of the resist pattern after development, one having no pattern collapse and having good rectangularity was rated as “good”, and the other was rated as “poor”. The smallest line width having no pattern collapse and having good rectangularity as the result of observation was used as the mark for the evaluation of resolution. Further, the minimum amount of electron beam energy capable of lithographing a good pattern shape was used as the mark for the evaluation of sensibility. The results are shown in Table 10.

TABLE 10

| | Resist auxiliary | Resolution | Sensibility | Resist |
|---|---|---|---|---|
| | film composition | (nmL/S) | (μC/cm²) | pattern shape |
| Example 1d | With | 40 | 10 | Good |
| Comparative | Without | 81 | 25 | Poor |
| Example 1d | | | | |

As is apparent from Table 10, the resist pattern in Example 1d was demonstrated to be significantly excellent in both resolution and sensibility, as compared with that in Comparative Example 1d. It was considered that such a result is due to the influence of the resist auxiliary film composition that increases the adhesiveness of the resist pattern. In addition, it was demonstrated in Example 1d that the resist pattern shape after development had no pattern collapse and had good rectangularity. Further, the difference in the resist pattern shapes after development indicated that the resist auxiliary film composition in Example 1d had good adhesiveness to the resist material.

Thus, when the resist auxiliary film composition that satisfies the requirement of the present embodiment is used, a good resist pattern shape can be obtained as compared with Comparative Example 1d, which does not satisfy the requirement. Also, resist auxiliary film compositions other than those described in Examples exhibit the same effect, as long as the above requirement of the present embodiment is satisfied.

### Example 2d

The resist auxiliary film composition prepared in Example 1d was applied to a SiO₂ substrate having a film thickness of 300 nm, baked at 240° C. for 60 seconds, and further baked at 400° C. for 120 seconds to form a resist underlayer film having a film thickness of 90 nm. A material of a silicon-containing intermediate layer was applied to the resist underlayer film and baked at 200° C. for 60 seconds to form a resist intermediate layer film having a film thickness of 35 nm. Further, the resist solution for ArF was applied to the resist intermediate layer film and baked at 130° C. for 60 seconds to form a photoresist layer having a film thickness of 150 nm. As the silicon-containing intermediate layer material, the silicon atom-containing polymer (polymer 1) described in <Synthetic Example 1> of Japanese Patent Laid-Open No. 2007-226170 was used.

Then, the above photoresist layer was exposed through a mask using an electron beam lithography apparatus (manufactured by ELIONIX INC.: ELS-7500, 50 keV), baked (PEB) at 115° C. for 90 seconds, and developed with a 2.38% by mass aqueous tetramethylammonium hydroxide (TMAH) solution for 60 seconds to obtain a positive type resist pattern of 45 nmL/S (1:1).

Thereafter, dry etching processing of the silicon-containing resist intermediate layer film was performed using “RIE-10NR” manufactured by Samco International, Inc. through the obtained resist pattern as a mask. Subsequently, dry etching processing of the resist underlayer film through the obtained silicon-containing resist intermediate layer film pattern as a mask and dry etching processing of the SiO₂ film through the obtained resist underlayer film pattern as a mask were sequentially performed.

Respective etching conditions were as follows.
Etching conditions of resist pattern on resist intermediate layer film
   Output: 50 W
   Pressure: 20 Pa
   Time: 1 min
   Etching gas
   Ar gas flow rate:CF₄ gas flow rate:O₂ gas flow rate=50:8:2 (sccm)
Etching conditions of resist intermediate layer film pattern on resist underlayer film
   Output: 50 W
   Pressure: 20 Pa
   Time: 2 min
   Etching gas
   Ar gas flow rate:CF₄ gas flow rate:O₂ gas flow rate=50:5:5 (sccm)
Etching conditions of resist underlayer film pattern on SiO₂ film
   Output: 50 W
   Pressure: 20 Pa
   Time: 2 min
   Etching gas

Ar gas flow rate:CsF₁₂ gas flow rate:C₂F₆ gas flow rate:O₂ gas flow rate=50:4:3:1 (sccm)

### <Evaluation of Pattern Shape>

A cross-section of the pattern of Example 2d obtained as described above (the shape of the SiO₂ film after etching) was observed using an electron microscope “S-4800” manufactured by Hitachi, Ltd. As a result, the shape of the SiO₂ film after etching in the multilayer resist processing was demonstrated to be rectangular with no defects and thus be good in Example, in which the resist auxiliary film composition of the present embodiment was used.

### [Resist Auxiliary Film Composition Containing Ethylenically Unsaturated Resin (0) and Acid Generating Agent]

Resist auxiliary film compositions were prepared according to the formulation shown in Table 11 and Table 12, and the solubility of the resins (i) to (v) and the acid generating agents (i) to (iv), which were used as materials, shown in Table 11 and Table 12 was evaluated.

### <Solvent>

HBM: methyl 2-hydroxyisobutyrate (manufactured by Mitsubishi Gas Chemical Company, Inc.)
αMBM: methyl α-methoxyisobutyrate (synthesized with reference to “US2014/0275016”)
αFBM: methyl α-formyloxyisobutyrate (synthesized with reference to “WO2020/004467”)
αABM: methyl α-acetyloxyisobutyrate (synthesized with reference to “WO2020/004466”)
3HBM: methyl 3-hydroxyisobutyrate (manufactured by Tokyo Kasei Kogyo Co., Ltd.)
iPHIB: isopropyl 2-hydroxyisobutyrate (manufactured by Mitsubishi Gas Chemical Company, Inc.)
PGME: 1-methoxy-2-propanol (manufactured by Sigma-Aldrich)

### <Resin>

The resins having the following compositional ratios (molecular weight) were synthesized by the above methods.
(i) EADM/NML=18/82 (Mn=3750)
(ii) MADM/NML=25/75 (Mn=2740)
(iii) MADM/GBLM=25/75 (Mn=3770)
(iv) MADM/NML/HADM=42/33/25 (Mn=7260)
(v) a copolymer having constitutional units of hydroxystyrene/t-butyl acrylate/styrene=3/1/1 (molar ratio) (manufactured by Maruzen Petrochemical Co., Ltd., Mw=12,000)

### <Acid Generating Agent>

(i) WPAG-336 (manufactured by FUJIFILM Wako Pure Chemical Corporation)
(ii) WPAG-367 (manufactured by FUJIFILM Wako Pure Chemical Corporation)
(iii) WPAG-145 (manufactured by FUJIFILM Wako Pure Chemical Corporation)
(iv) triphenylsulfonium trifluoro-1-butanesulfonate (Sigma-Aldrich)

Each resin of the kind shown in Table 11 was added to each solvent of the kind shown in Table 11 such that the resin concentration was 15 wt %, and each acid generating agent of the kind shown in Table 11 was added to the mixture such that the acid generating agent concentration was 1 wt %, to prepare each of the resist auxiliary film compositions of Examples A1-1 to A1-4 and Comparative Example A1-1. The state after stirring at room temperature for 24 hours was visually evaluated according to the following criteria.
Rank S: Dissolved (clear solution was visually found)
Rank A: Almost dissolved (almost clear solution was visually found)
Rank C: Insoluble (cloudy solution was visually found)

Each resin shown in Table 12 was added to each solvent shown in Table 12 such that the resin concentration was 40 wt %, and each acid generating agent of the kind shown in Table 12 was added to the mixture such that the acid generating agent concentration was a predetermined concentration, to prepare each of the resist auxiliary film compositions of Examples A2-1a to A2-5d and Comparative Example A2-1. The state after stirring at room temperature for 1 hour was visually evaluated according to the following criteria.
Rank S: 5 wt % Dissolved (clear solution was visually found)
Rank A: 1 wt % Dissolved (clear solution was visually found)
Rank C: 1 wt % Insoluble (cloudy solution was visually found)

The results are shown in Table 11 and Table 12.

TABLE 11

| | | Resin | Resin | Resin | Resin |
|---|---|---|---|---|---|
| | | (i) | (ii) | (iii) | (iv) |
| | | Acid | Acid | Acid | Acid |
| | | generating | generating | generating | generating |
| | | agent | agent | agent | agent |
| | Solvent | (iv) | (iv) | (iv) | (iv) |
| Example A1-1 | HBM/αMBM | A | A | S | S |
| | (1:1 | | | | |
| | weight ratio) | | | | |
| Example A1-2 | HBM/αFBM | S | S | S | S |
| | (1:1 | | | | |
| | weight ratio) | | | | |
| Example A1-3 | HBM/3HBM | A | A | S | S |
| | (1:1 | | | | |
| | weight ratio) | | | | |
| Example A1-4 | HBM | A | A | S | S |
| Example A1-5 | HBM/PGME | A | A | S | S |
| | (1:1 | | | | |
| | weight ratio) | | | | |
| Comparative | PGMEA | C | C | C | C |
| Example A1-1 | | | | | |

TABLE 12-1

| | | Resin | Resin | Resin |
|---|---|---|---|---|
| | | (v) | (v) | (v) |
| | | Acid | Acid | Acid |
| | | generating | generating | generating |
| | | agent | agent | agent |
| | Solvent | (i) | (ii) | (iii) |
| Example A2-1a | HBM/αMBM | S | S | S |
| | (1:1 | | | |
| | weight ratio) | | | |
| Example A2-1b | HBM/αMBM | S | S | S |
| | (9:1 | | | |
| | weight ratio) | | | |
| Example A2-1c | HBM/αMBM | S | S | S |
| | (1:0.001 | | | |
| | weight ratio) | | | |
| Example A2-1d | HBM/αMBM | S | S | S |
| | (1:0.00005 | | | |
| | weight ratio) | | | |
| Example A2-2a | HBM/αFBM | S | S | S |
| | (1:1 | | | |
| | weight ratio) | | | |
| Example A2-2b | HBM/αFBM | S | S | S |
| | (9:1 | | | |
| | weight ratio) | | | |

TABLE 12-2

| | | | | |
|---|---|---|---|---|
| Example A2-2c | HBM/αFBM | S | S | S |
| | (1:0.001 weight ratio) | | | |
| Example A2-2d | HBM/αFBM | S | S | S |
| | (1:0.00005 weight ratio) | | | |
| Example A2-3a | HBM/3HBM | S | S | S |
| | (1:1 weight ratio) | | | |
| Example A2-3b | HBM/3HBM | S | S | S |
| | (9:1 weight ratio) | | | |
| Example A2-3c | HBM/3HBM | S | S | S |
| | (1:0.001 weight ratio) | | | |
| Example A2-3d | HBM/3HBM | S | S | S |
| | (1:0.00005 weight ratio) | | | |
| Example A2-4 | HBM | S | S | A |
| Example A2-5a | HBM/PGME | S | S | S |
| | (1:1 weight ratio) | | | |
| Example A2-5b | HBM/PGME | S | S | S |
| | (9:1 weight ratio) | | | |
| Example A2-5c | HBM/PGME | S | S | S |
| | (1:0.001 weight ratio) | | | |
| Example A2-5d | HBM/PGME | S | S | S |
| | (1:0.00005 weight ratio) | | | |
| Comparative | PGMEA | A | C | A |
| Example A2-1 | | | | |

It is found from Table 11 that the resist auxiliary film compositions prepared in Examples A1-1 to A1-5 are excellent in the solubility of the resin and can prepare various resist auxiliary film compositions as compared with the resist auxiliary film composition of Comparative Example A1-1. In particular, the resist auxiliary film compositions in which the solvent (B) contains αFBM as the solvent (B2) exhibit high solubility of any of the resins and are suitably used.

It is found from Table 12 that the resist auxiliary film compositions prepared in Examples A2-1a to A2-5d are excellent in the solubility of any acid generating agents as compared with the resist auxiliary film composition of Comparative Example A2-1, and that the resist auxiliary film composition can be thus prepared even when any of the acid generating agents is used. In particular, the resist auxiliary film compositions in which the solvent (B) contains αMBM, αFBM, 3HBM, or PGME as the solvent (B2) exhibit high solubility of any acid generating agents and are suitably used.

### [Resist Auxiliary Film Composition Containing Ethylenically Unsaturated Resin (0)]

A copolymer having constitutional units of hydroxystyrene/t-butyl acrylate/styrene=3/1/1 (molar ratio) as the ethylenically unsaturated resin (0) (manufactured by Maruzen Petrochemical Co., Ltd., Mw=12,000) was mixed with the solvent of the kind shown in Table 13 to prepare a resist auxiliary film composition having an active component (resin for KrF) concentration described in Table 13.

Then, a coating film was formed from the prepared resist auxiliary film composition on a silicon wafer by spin coating at 1500 rpm, and the coating film was subjected to prebaking at 140° C. for 60 seconds to form a resist auxiliary film. The film thicknesses were measured at randomly selected 5 points on the resist auxiliary film, and the average value of the film thicknesses at 5 points was calculated as the average film thickness, and the film thickness was evaluated. In addition, the difference between the maximum value and the minimum value of the film thicknesses was divided by the average value, and the result was evaluated as film uniformity. The results are shown in Table 13.
Film thicknesses:
   Rank A: 20 μm or more
   Rank B: 15 μm or more and less than 20 μm
   Rank C: less than 15 μm
Film uniformity:
   Rank A: less than 15
   Rank B: 15 or more and less than 30)
   Rank C: 30 or more

TABLE 13

| | | Concentration | Film | Film |
|---|---|---|---|---|
| | | wt % | thickness | uniformity |
| Example A3-1a | HBM/αMBM | 40 | B | A |
| | (1:1 weight ratio) | | | |
| Example A3-1b | HBM/αMBM | 45 | A | A |
| | (1:1 weight ratio) | | | |
| Example A3-1c | HBM/αMBM | 45 | A | A |
| | (1:0.001 weight ratio) | | | |
| Example A3-2a | HBM/αFBM | 40 | A | A |
| | (1:1 weight ratio) | | | |
| Example A3-2b | HBM/αFBM | 40 | A | B |
| | (1:2 weight ratio) | | | |
| Example A3-2c | HBM/αFBM | 40 | A | A |
| | (1:0.001 weight ratio) | | | |
| Example A3-3a | HBM/3HBM | 40 | B | A |
| | (1:1 weight ratio) | | | |
| Example A3-3b | HBM/3HBM | 40 | B | A |
| | (9:1 weight ratio) | | | |
| Example A3-3c | HBM/3HBM | 40 | B | A |
| | (1:0.001 weight ratio) | | | |
| Example A3-4a | HBM | 40 | B | A |
| Example A3-4b | HBM | 45 | B | B |
| Example A3-5a | HBM/PGME | 40 | B | A |
| | (1:1 weight ratio) | | | |
| Example A3-5b | HBM/PGME | 45 | B | A |
| | (9:1 weight ratio) | | | |
| Example A3-5c | HBM/PGME | 45 | B | A |
| | (1:0.001 weight ratio) | | | |
| Comparative | PGMEA | 40 | C | — |
| Example A3-1a | | | | |
| Comparative | PGMEA | 45 | C | — |
| Example A3-1b | | | | |

It is found from Table 13 that the resist auxiliary film compositions prepared in Examples A3-1a to A3-5c can form thick resist auxiliary films as compared with the resist auxiliary film compositions of Comparative Examples A3-1a to A3-1b. In particular, the resist auxiliary film compositions in which the solvent (B) contains αMBM, αFBM, 3HBM, or PGME as the solvent (B2) are excellent in film uniformity and suitably used. In addition, the resist auxiliary film compositions containing αFBM can have a film thickness of 20 μm or more and are suitably used when the resin concentration is 40 wt %. Further, the resist auxiliary film compositions containing αMBM can have a film thickness of 20 μm or more and are suitably used when the resin concentration is 45 wt %.

### <Evaluation of In-Plane Uniformity of Resist Auxiliary Film Composition>

The resin for KrF (copolymer having constitutional units of hydroxystyrene/t-butyl acrylate/styrene=3/1/1 (molar ratio) (manufactured by Maruzen Petrochemical Co., Ltd., Mw=12,000)) was mixed with the solvent of the kind shown in Table 14 to prepare a resist auxiliary film composition having an active component (resin for KrF) concentration described in Table 14.

Then, a coating film was formed from the prepared resist auxiliary film composition on a silicon wafer at a main spin of 1200 rpm, and the coating film was subjected to prebaking at 110° C. for 90 seconds to form a resist auxiliary film having an average film thickness of 7.2 μm. Film thicknesses were measured at 50 points positioned at a distance of 3 mm in the diameter direction on the resist auxiliary film. A triple of the standard deviation of the film thickness was divided by the average film thickness to calculate the film thickness unevenness 3σ, and the in-plane uniformity was evaluated. The results are shown in Table 14.
In-plane uniformity:
   Rank A: 3σ≤less than 0.02
   Rank B: 0.02 or more and less than 0.04
   Rank C: 0.04 or more

TABLE 14

| | | Concentration | In-plane |
|---|---|---|---|
| | | wt % | uniformity |
| Example A4-1a | HBM/PGME | 30.9 | A |
| | (3:7 weight ratio) | | |
| Example A4-1b | HBM/PGME | 30.1 | A |
| | (7:3 weight ratio) | | |
| Example A4-2a | HBM | 29.4 | B |
| Comparative | PGME | 31.8 | C |
| Example A4-1a | | | |

### [Resist Pattern Evaluation]

### (Preparation of Resist Auxiliary Film Composition)

Each resist auxiliary film composition was prepared so as to have the formulation shown in Table 15. The following were used as the polymer, the acid generating agent, the cross-linking agent, and the organic solvent.

Polymer: The resin of the formula (R1-1) was prepared as follows. Specifically, 30 g of 4,4-biphenol, 15 g of 4-biphenylaldehyde, and 100 mL of butyl acetate were blended, and 3.9 g of p-toluenesulfonic acid was added thereto to prepare a reaction solution. The reaction solution was reacted by stirring at 90° C. for 3 hours. Then, the reaction solution was concentrated, and the reaction solution was added dropwise into 400 mL of n-heptane. In this manner, the obtained resin product was solidified and purified, and the produced white powder was collected by filtering and dried at 40° C. under reduced pressure overnight to obtain a resin represented by the following formula (R1-1).

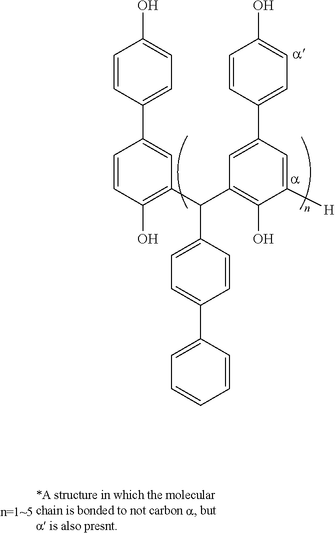

Acid generating agent: di-tertiary butyl diphenyliodonium nonafluoromethanesulfonate (DTDPI) manufactured by Midori Kagaku Co., Ltd.
Cross-linking agent: NIKALACMX270 (NIKALAC) manufactured by Sanwa Chemical Co., Ltd.
TMOM-BP (a compound represented by the following formula) manufactured by Honshu Chemical Industry Co., Ltd.

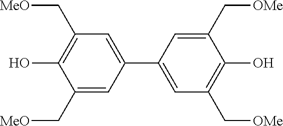 [SMILES: COCc1cc(-c2cc(COC)c(O)c(COC)c2)cc(COC)c1O]

Organic solvent: methyl 2-hydroxyisobutyrate (HBM)
   methyl α-methoxyisobutyrate (αMBM)
   methyl α-formyloxyisobutyrate (αFBM)
   methyl 3-hydroxyisobutyrate (3HBM)
   isopropyl 2-hydroxyisobutyrate (iPHIB)
   1-methoxy-2-propanol (PGME)

TABLE 15-1

| | | | Organic | | Cross-linking |
|---|---|---|---|---|---|
| | Polymer | Organic solvent | solvent (B2) | Acid generating | agent |
| | (parts by | (B1) | (parts by | agent | (parts by |
| | mass) | (parts by mass) | mass) | (parts by mass) | mass) |
| Example | R1-1 | HBM | αMBM | DTDPI | TMOM-BP |
| A5-1 | (10) | (45) | (45) | (0.5) | (0.5) |
| Example | R1-1 | HBM | αFBM | DTDPI | TMOM-BP |
| A5-2 | (10) | (45) | (45) | (0.5) | (0.5) |
| Example | R1-1 | HBM | 3HBM | DTDPI | TMOM-BP |
| A5-3 | (10) | (45) | (45) | (0.5) | (0.5) |
| Example | R1-1 | HBM | — | DTDPI | TMOM-BP |
| A5-4 | (10) | (90) | | (0.5) | (0.5) |
| Example | R1-1 | iPHIB | — | DTDPI | TMOM-BP |
| A5-5 | (10) | (90) | | (0.5) | (0.5) |
| Example | R1-1 | iPHIB | 3HBM | DTDPI | TMOM-BP |
| A5-6 | (10) | (45) | (45) | (0.5) | (0.5) |
| Example | R1-1 | HBM | αFBM | DTDPI | TMOM-BP |
| A5-7 | (10) | (45) | (45) | (0.5) | (0.5) |
| Example | R1-1 | HBM | αMBM | DTDPI | TMOM-BP |
| A5-8 | (10) | (89) | (1) | (0.5) | (0.5) |
| Example | R1-1 | HBM | αMBM | DTDPI | TMOM-BP |
| A5-9 | (10) | (89.995) | (0.005) | (0.5) | (0.5) |
| Example | R1-1 | HBM | αFBM | DTDPI | TMOM-BP |
| A5-10 | (10) | (89) | (1) | (0.5) | (0.5) |
| Example | R1-1 | HBM | αFBM | DTDPI | TMOM-BP |
| A5-11 | (10) | (89.995) | (0.005) | (0.5) | (0.5) |
| Example | R1-1 | HBM | 3HBM | DTDPI | TMOM-BP |
| A5-12 | (10) | (89) | (1) | (0.5) | (0.5) |
| Example | R1-1 | HBM | 3HBM | DTDPI | TMOM-BP |
| A5-13 | (10) | (89.995) | (0.005) | (0.5) | (0.5) |
| Example | R1-1 | HBM | PGME | DTDPI | TMOM-BP |
| A5-14 | (10) | (45) | (45) | (0.5) | (0.5) |

TABLE 15-2

| Example | R1-1 | HBM | PGME | DTDPI | TMOM-BP |
|---|---|---|---|---|---|
| A5-15 | (10) | (89) | (1) | (0.5) | (0.5) |
| Example | R1-1 | HBM | PGME | DTDPI | TMOM-BP |
| A5-16 | (10) | (89.995) | (0.005) | (0.5) | (0.5) |

Then, the resist auxiliary film composition prepared in Examples AS-1 to AS-16 was applied to a SiO₂ substrate having a film thickness of 300 nm, baked at 240° C. for 60 seconds, and further baked at 400° C. for 120 seconds to form a resist underlayer film having a film thickness of 85 nm. A resist solution for ArF was applied to each resist underlayer film, and baked at 130° C. for 60 seconds to form a photoresist layer having a film thickness of 140 nm.

As the resist solution for ArF, a solution prepared by blending 5 parts by mass of the resin of the following formula (1d), 1 part by mass of triphenylsulfonium nonafluoromethanesulfonate, 2 parts by mass of tributylamine, and 92 parts by mass of PGMEA was used.

The resin of the following formula (1d) was prepared as follows. Specifically, 4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile were dissolved in 80 mL of tetrahydrofuran to obtain a reaction solution. The reaction solution was polymerized for 22 hours by holding the reaction temperature at 63° C. under nitrogen atmosphere, and the reaction solution was added dropwise into 400 mL of n-hexane. In this manner, the obtained resin product was solidified and purified, and the produced white powder was collected by filtering and dried at 40° C. under reduced pressure overnight to obtain a resin represented by the following formula (1d).

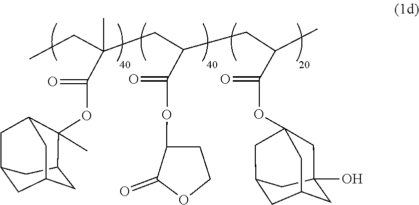

(In the formula (1d), 40, 40, and 20 indicate the ratio between the constitutional units, and do not indicate the block copolymer.)

Then, the above photoresist layer was exposed using an electron beam lithography apparatus (manufactured by ELIONIX INC.; ELS-7500, 50 keV), baked (PEB) at 115° C. for 90 seconds, and developed with a 2.38% by mass aqueous tetramethylammonium hydroxide (TMAH) solution for 60 seconds to obtain a positive type resist pattern.

### Comparative Example A4

A positive type resist pattern was obtained in the same manner as in Example A5-1, except that no resist underlayer film was formed, or in other words, a photoresist layer was directly formed on a SiO₂ substrate.

### [Evaluation]

For each of Examples A5-1 to A5-16 and Comparative Example A5, the shapes of the obtained resist patterns with 40 nm L/S (1:1) and 80 nm L/S (1:1) were observed using an electron microscope “S-4800” manufactured by Hitachi, Ltd. Regarding the shape of the resist pattern after development, one having no pattern collapse and having good rectangularity was rated as “good”, and the other was rated as “poor”. The smallest line width having no pattern collapse and having good rectangularity as a result of observation was used as the mark for the evaluation of resolution. Further, the minimum amount of electron beam energy capable of lithographing a good pattern shape was used as the mark for the evaluation of sensibility. The results are shown in Table 16.

TABLE 16

| | Resist auxiliary | Resolution | Sensibility | Resist pattern |
|---|---|---|---|---|
| | film composition | (nmL/S) | (μC/cm²) | shape |
| Example A5-1 | With | 40 | 10 | Good |
| Example A5-2 | With | 40 | 10 | Good |
| Example A5-3 | With | 40 | 10 | Good |
| Example A5-4 | With | 40 | 10 | Good |
| Example A5-5 | With | 40 | 10 | Good |
| Example A5-6 | With | 40 | 10 | Good |
| Example A5-7 | With | 40 | 10 | Good |
| Example A5-8 | With | 40 | 10 | Good |
| Example A5-9 | With | 40 | 10 | Good |
| Example A5-10 | With | 40 | 10 | Good |
| Example A5-11 | With | 40 | 10 | Good |
| Example A5-12 | With | 40 | 10 | Good |
| Example A5-13 | With | 40 | 10 | Good |
| Example A5-14 | With | 40 | 10 | Good |
| Example A5-15 | With | 40 | 10 | Good |
| Example A5-16 | With | 40 | 10 | Good |
| Comparative | Without | 81 | 25 | Poor |
| Example A5 | | | | |

As is apparent from Table 16, the resist patterns in Examples A5-1 to A5-16 were demonstrated to be significantly excellent in both resolution and sensibility, as compared with that in Comparative Example A5. It was considered that such a result is due to the influence of the resist auxiliary film composition that increases the adhesiveness of the resist pattern. In addition, it was demonstrated in Examples A5-1 to A5-16 that the resist pattern shapes after development had no pattern collapse and had good rectangularity. Further, the difference in the resist pattern shapes after development indicated that the resist auxiliary film compositions in Examples A5-1 to A5-16 had good adhesiveness to the resist material.

Thus, when the resist auxiliary film composition that satisfies the requirement of the present embodiment is used, a good resist pattern shape can be obtained as compared with Comparative Example A5, which does not satisfy the requirement. Also, other resist auxiliary film compositions other than those described in Examples exhibit the same effect, as long as the above requirement of the present embodiment is satisfied.

### Examples A6-1 to A6-16

The resist auxiliary film composition prepared in Examples A5-1 to A5-16 was applied to a SiO₂ substrate having a film thickness of 300 nm, baked at 240° C. for 60 seconds, and further baked at 400° C. for 120 seconds to form a resist underlayer film having a film thickness of 90 nm. A material of silicon-containing intermediate layer was applied to the resist underlayer film and baked at 200° C. for 60 seconds to form a resist intermediate layer film having a film thickness of 35 nm. Further, the resist solution for ArF was applied to the resist intermediate layer film and baked at 130° C. for 60 seconds to form a photoresist layer having a film thickness of 150 nm. As the silicon-containing intermediate layer material, the silicon atom-containing polymer (polymer 1) described in <Synthetic Example 1> of Japanese Patent Laid-Open No. 2007-226170 was used.

Then, the above photoresist layer was exposed through a mask using an electron beam lithography apparatus (manufactured by ELIONIX INC.: ELS-7500, 50 keV), baked (PEB) at 115° C. for 90 seconds, and developed with a 2.38% by mass aqueous tetramethylammonium hydroxide (TMAH) solution for 60 seconds to obtain a positive type resist pattern of 45 nmL/S (1:1).

Thereafter, dry etching processing of the silicon-containing resist intermediate layer film was performed using “RIE-10NR” manufactured by Samco International, Inc. through the obtained resist pattern as a mask. Subsequently, dry etching processing of the resist underlayer film through the obtained silicon-containing resist intermediate layer film pattern as a mask and dry etching processing of the SiO₂ film through the obtained resist underlayer film pattern as a mask were sequentially performed.

Respective etching conditions were as follows.
Etching conditions of resist pattern on resist intermediate layer film
   Output: 50 W
   Pressure: 20 Pa
   Time: 1 min
   Etching gas
   Ar gas flow rate:CF₄ gas flow rate:O₂ gas flow rate=50:8:2 (sccm)
Etching conditions of resist intermediate layer film pattern on resist underlayer film
   Output: 50 W
   Pressure: 20 Pa
   Time: 2 min
   Etching gas
   Ar gas flow rate:CF₄ gas flow rate:O₂ gas flow rate=50:5:5 (sccm)
Etching conditions of resist underlayer film pattern on SiO₂ film
   Output: 50 W
   Pressure: 20 Pa
   Time: 2 min
   Etching gas
   Ar gas flow rate:CsF₁₂ gas flow rate:C₂F₆ gas flow rate:O₂ gas flow rate=50:4:3:1 (sccm)

### <Evaluation of Pattern Shape>

A cross-section of each of the patterns of Examples A6-1 to A6-11 and A6-14 to A6-16 obtained as described above (the shape of the SiO₂ film after etching) was observed using an electron microscope “S-4800” manufactured by Hitachi, Ltd. As a result, the shape of the SiO₂ film after etching in the multilayer resist processing was demonstrated to be rectangular with no defects and thus be good in Examples, in which the resist auxiliary film composition of the present embodiment was used.

### [Evaluation of Stepped Substrate Embedding Properties]

The evaluation of the embedding properties on a stepped substrate was performed by the following procedure.

The resist auxiliary film compositions prepared in Examples A5-1 to A5-6 and A5-14 and the resist auxiliary film composition A7 described below was applied to a SiO₂ substrate having a film thickness of 150 nm with a line and space of 60 nm, and baked at 400° C. for 60 seconds to form a resist underlayer film having a film thickness of 100 nm. The cross-section of the obtained resist underlayer film was cut out and observed with an electron microscope, and the embedding properties on the stepped substrate were evaluated. The results are shown in Table 16.

### <Evaluation Criteria>

A: Projected and recessed portions of the SiO₂ substrate with a line and space of 60 nm have no defects, and the resist underlayer film is embedded therein.
C: Projected and recessed portions of SiO₂ substrate with a line and space of 60 nm have defects, so that the resist underlayer film is not embedded therein.

### [Evaluation of Flatness]

The resist auxiliary film compositions obtained above was applied to a SiO₂ stepped substrate in which trenches with a width of 100 nm, a pitch of 150 nm, and a depth of 150 nm (aspect ratio: 1.5) and trenches with a width of 5 μm and a depth of 150 nm (open space) coexisted. Thereafter, the substrate was calcined at 400° C. for 120 seconds in the atmosphere to form each resist underlayer film having a film thickness of 100 nm. The shape of the resist underlayer film was observed by a scanning electron microscope (“S-4800” manufactured by Hitachi High-Technologies Corporation), and the difference (ΔFT) between the maximum value and the minimum value of the film thicknesses of the resist underlayer film on the trench or space was measured. The results are shown in Table 17.

### <Evaluation Criteria>

S: ΔFT<10 nm (best flatness)
A: 10 nm≤ΔFT<20 nm (good flatness)
B: 20 nm≤ΔFT<40 nm (slightly good flatness)
C: 40 nm≤ΔFT (poor flatness)

TABLE 17

| | Resist auxiliary | Step embedding | |
|---|---|---|---|
| | film composition | properties | Flatness |
| Example A7-1 | Composition of | A | A |
| | Example A5-1 | | |
| Example A7-2 | Composition of | A | A |
| | Example A5-2 | | |
| Example A7-3 | Composition of | A | S |
| | Example A5-3 | | |
| Example A7-4 | Composition of | A | A |
| | Example A5-4 | | |
| Example A7-5 | Composition of | A | S |
| | Example A5-5 | | |
| Example A7-6 | Composition of | A | S |
| | Example A5-6 | | |
| Example A7-7 | Composition of | A | A |
| | Example A5-14 | | |
| Comparative | Resist auxiliary | C | C |
| Example A7 | film composition | | |
| | A7 | | |

### (Preparation of Resist Auxiliary Film Composition A7)

The resist auxiliary film composition A7 was prepared in the same manner as in Example A5-4, except that the solvent was changed from HBM to 1-methoxy-2-propanol (PGME).

### <Evaluation of Step Embedding Properties and Flatness>

The step embedding properties and flatness of Examples A7-1 to A7-7 obtained as described above were demonstrated to be good. In particular, the resist auxiliary film compositions in which the solvent (B) contains 3HBM as the solvent (B2), or the resist auxiliary film compositions in which the solvent (B) contains iPHIB as the solvent (B1) are excellent in both step embedding properties and flatness and suitably used.

Resist auxiliary film compositions other than those described in Examples also exhibit the same effect, as long as the above requirement of the present embodiment is satisfied.

1. A resist auxiliary film composition comprising:
(A) a resin, and
(B) a solvent comprising: (B1) a compound represented by the following general formula (b-1), wherein
a content of an active component is 45% by mass or less based on the total amount of the resist auxiliary film composition: 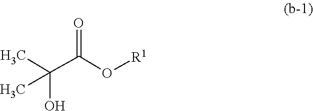
wherein R¹ is an alkyl group having 1 to 10 carbon atoms.

2. The resist auxiliary film composition according to claim 1, further comprising: (C) at least one additive selected from the group consisting of a photosensitizer and an acid generating agent.

3. The resist auxiliary film composition according to claim 1, wherein R¹ in the general formula (b-1) is a methyl group, an ethyl group, an n-propyl group, an i-propyl group, an n-butyl group, an i-butyl group, an s-butyl group, or a t-butyl group.

4. The resist auxiliary film composition according to claim 1, wherein R¹ in the general formula (b-1) is an ethyl group, an n-propyl group, an i-propyl group, an n-butyl group, an i-butyl group, an s-butyl group, or a t-butyl group.

5. The resist auxiliary film composition according to claim 1, wherein the solvent (B) comprises: (B2) a solvent other than the compound (B1).

6. The resist auxiliary film composition according to claim 5, wherein the solvent (B) comprises one or more selected from the group consisting of methyl α-methoxyisobutyrate, methyl α-formyloxyisobutyrate, methyl α-acetyloxyisobutyrate, methyl 3-hydroxyisobutyrate, and 1-methoxy-2-propanol, as the solvent (B2).

7. The resist auxiliary film composition according to claim 5, wherein the solvent (B) comprises one or more selected from the group consisting of methyl α-methoxyisobutyrate, methyl α-formyloxyisobutyrate, methyl α-acetyloxyisobutyrate, and methyl 3-hydroxyisobutyrate, as the solvent (B2).

8. The resist auxiliary film composition according to claim 5, wherein the solvent (B2) is contained in an amount of 100% by mass or less based on the total amount (100% by mass) of the compound (B1).

9. The resist auxiliary film composition according to claim 8, wherein the solvent (B2) is contained in an amount of 0.0001% by mass or more based on the total amount (100% by mass) of the compound (B1).

10. The resist auxiliary film composition according to claim 5, wherein the solvent (B2) is contained in an amount less than 100% by mass based on the total amount (100% by mass) of the resist auxiliary film composition.

11. The resist auxiliary film composition according to claim 1, wherein the resin (A) comprises a novolac resin (A1).

12. The resist auxiliary film composition according to claim 1, wherein the resin (A) comprises an ethylenically unsaturated resin (A2).

13. The resist auxiliary film composition according to claim 1, wherein the resin (A) comprises a high carbon resin (A3).

14. The resist auxiliary film composition according to claim 1, wherein the resin (A) comprises a silicon-containing resin (A4).

15. The resist auxiliary film composition according to claim 1, wherein the resist auxiliary film is a resist underlayer film.

16. The resist auxiliary film composition according to claim 1, wherein the resist auxiliary film is a resist intermediate layer film.

17. A method for forming a pattern, comprising:
forming a resist underlayer film on a substrate by using the resist auxiliary film composition according to claim 15,
forming at least one photoresist layer on the resist underlayer film, and
irradiating a predetermined region of the photoresist layer with radiation after the forming of the at least one photoresist layer, followed by developing.

18. A method for forming a pattern, comprising:
forming a resist underlayer film on a substrate by using the resist auxiliary film composition according to claim 15,
forming a resist intermediate layer film on the resist underlayer film,
forming at least one photoresist layer on the resist intermediate layer film,
irradiating a predetermined region of the photoresist layer with radiation after the forming of the at least one photoresist layer, followed by developing to form a resist pattern, and
etching the resist intermediate layer film by using the resist pattern as a mask after the irradiating of the predetermined region of the photoresist layer, etching the resist underlayer film by using the obtained resist intermediate layer film pattern as an etching mask, and etching the substrate by using the obtained resist underlayer film pattern as an etching mask to form a pattern on the substrate.

19. A method for forming a pattern, comprising:
forming a resist underlayer film on a substrate,
forming a resist intermediate layer film on the resist underlayer film by using the resist auxiliary film composition according to claim 16,
forming at least one photoresist layer on the resist intermediate layer film,
irradiating a predetermined region of the photoresist layer with radiation after the forming of the at least one photoresist layer, followed by developing to form a resist pattern, and
etching the resist intermediate layer film by using the resist pattern as a mask after the irradiating of the predetermined region of the photoresist layer, etching the resist underlayer film by using the obtained resist intermediate layer film pattern as an etching mask, and etching the substrate by using the obtained resist underlayer film pattern as an etching mask to form a pattern on the substrate.
